(19) United States
(12) Patent Application Publication
(10) Pub. No.: US 2012/0184517 A1
(43) Pub. Date: Jul. 19, 2012

(54) TREATMENT OF DISEASES WITH ALTERED SMOOTH MUSCLE CONTRACTILITY

(76) Inventors: Steven Marx, Scarsdale, NY (US); Jeanine D'Armiento, New York, NY (US); Andrew Marks, Larchmont, NY (US)

(21) Appl. No.: 13/266,794

(22) PCT Filed: Apr. 30, 2010
(86) PCT No.: PCT/US10/01288

§ 371 Date: Mar. 19, 2012

Related U.S. Application Data
(60) Provisional application No. 61/214,948, filed on Apr. 30, 2009.

(51) Int. Cl. A61K 31/56; A61P 11/06; A61P 21/00; A61P 9/12; A61P 13/10; A61P 29/00; A61K 31/352; A61P 11/00

(52) U.S. Cl. 514/171; 514/456

(57) ABSTRACT

The present invention provides, inter alia, methods and compositions for treating or ameliorating the effects of a disease characterized by altered smooth muscle contractility, such as e.g., asthma.

### CROSS-REFERENCE TO RELATED APPLICATIONS

This application claims benefit to U.S. Provisional Patent Application Ser. No. 61/214,948, filed Apr. 30, 2009, the entire content of which is hereby incorporated by reference as if recited in full herein.

### GOVERNMENT FUNDING

This invention was made with government support under P01 HL081172 awarded by the National Heart, Lung and Blood Institute of the National Institutes of Health. The government has certain rights in the invention.

### FIELD OF THE INVENTION

The present invention relates, inter alia, to pharmaceutical compositions and methods to treat or ameliorate the effects of diseases characterized by altered smooth muscle contractility, such as e.g., asthma.

### BACKGROUND OF THE INVENTION

Asthma-associated airway hyperresponsiveness (AHR) is primarily mediated by excessive airway smooth muscle (ASM) cell contraction, yet the mechanisms responsible for this behavior are not clearly elucidated. Although asthma involves inflammation, ASM cell hypertrophy and hyperplasia, the primary event leading to AHR is the stimulation of ASM cell contraction. Despite current therapy (anti-cholinergics, anti-histamines, anti-leukotrienes, β-agonists and phosphodiesterase inhibitors), many asthmatic patients suffer from airway hyperreactivity. In addition, side-effects from these drugs can also limit their efficacy. Thus, novel approaches to treat asthma may have a profound impact on improving the morbidity of this disease. Regulating the growth and contractility of ASM represents an important target for the treatment of asthma.

The elevation of intracellular calcium, [Ca²⁺]ᵢ, which may occur in asthma (Black et al., Intrinsic asthma: is it intrinsic to the smooth muscle? Clin Exp Allergy (2009); Kellner et al., Mechanisms altering airway smooth muscle cell Ca+ homeostasis in two asthma models. Respiration 76:205-215. (2008)), plays a critical role in airway smooth muscle (ASM) contractility and may also affect cell proliferation. The increase in Ca²⁺ can be achieved in two ways: (a) release of Ca²⁺ from the internal stores of the SR and/or (b) Ca²⁺ influx from the extracellular space via plasma membrane ion channels. Contraction of smooth muscle is triggered by phosphorylation of myosin, catalyzed by Ca²⁺/calmodulin-dependent myosin light chain kinase (MLCK), which is activated by Ca²⁺. In airway and vascular SMCs agonists initiate, but cannot maintain, contraction in Ca²⁺-free conditions, which indicates that internal stores require refilling by Ca²⁺ influx. The Ca²⁺ influx may be mediated by voltage-dependent and voltage-independent mechanisms. The contractility of smooth muscle is regulated by a feed-back mechanism whereby the localized, transient increase in cytoplasmic Ca²⁺ concentration due to activation of sarcoplasmic reticular (SR) ryanodine receptors (RyR) activates plasma membrane BK channels (large conductance voltage- and Ca²⁺-activated K⁺ channels). The activation of BK channels causes transient membrane hyperpolarization, inhibition of Ca²⁺ influx through voltage-dependent Ca²⁺ channels, reduced intracellular Ca²⁺ concentration ([Ca²⁺]ᵢ and a subsequent decrease in smooth muscle tension.

Furthermore, the high incidence of stroke and hypertension in the United States remains a leading indication for visits to physicians, the use of prescription drugs and morbidity/mortality. It is estimated that more than 50 million Americans (approximately one third of the adult population) currently suffer from hypertension. Two-thirds of the population over age 70 suffers from hypertension. Chronic blood pressure elevation leads to end-organ damage, including eye, cardiac, and central nervous system damage. Thus, greater understanding of the molecular mechanisms leading to the regulation of membrane excitability may have important implications in improving therapeutic modalities.

The large-conductance Ca²⁺-activated K⁺ (BK_{Ca}) channel complex plays a critical role in regulating contractile tone in smooth muscle and the vasculature (Brenner, et al., Vasoregulation by the β1 subunit of the calcium-activated potassium channel. Nature, 2000. 407(6806):870-6; Brayden, et al., Regulation of arterial tone by activation of calcium-dependent potassium channels. Science, 1992. 256(5056):532-5). Furthermore, neuronal BK_{Ca} channel function is not well-studied, yet it remains clear that the channel has significant effects on neurotransmitter release and neuronal discharges (Robitaille, et al., Functional colocalization of calcium and calcium-gated potassium channels in control of transmitter release. Neuron, 1993. II(4):645-55). Thus, the BK_{Ca} channel represents an important integrator of signal transduction pathways, potently mediating cellular excitability in a diverse group of cell types. Recent studies have suggested that the channel may have a role in innate immunity in neutrophils (Ahluwalia, et al., The large-conductance Ca²⁺-activated K⁺ channel is essential for innate immunity. Nature, 2004. 427(6977):853-8), recognition as a heme-binding protein (Tang, et al., Haem can bind to and inhibit mammalian calcium-dependent Slo1 BK channels. Nature, 2003. 425(6957):531-5), behavior responses to ethanol (Davies, et al., A central role of the BK potassium channel in behavioral responses to ethanol in C. elegans. Cell, 2003. 115(6):655-66) and function as a protective mechanism against ischemically-driven cell death in cardiac myocytes (Xu, et al., Cytoprotective role of Ca²⁺-activated K⁺ channels in the cardiac inner mitochondrial membrane. Science, 2002. 298(5595):1029-33). These novel functions of the BK_{Ca} channel remain to be further validated and explored (Mocaydlowski, E. G., BK Channel News: Full Coverage on the Calcium Bowl. J. Gen. Physiol, 2004. 123(5):471-3). Utilizing molecular biologic and electrophysiologic approaches, the present inventors are seeking to elucidate the mechanism(s) through which the BK_{Ca} channel is allosterically regulated.

By unraveling the complex mechanism(s) mediating phosphorylation-dependent and β1 subunit regulation of the channel, the present inventors seek to identify specific regions that are responsible for activation and inhibition of channel function. Novel approaches for the treatment of disorders ranging from neurologic dysfunction (seizures, memory) to vascular complications related to diabetes or hypertension will follow from elucidation of the basic mechanism(s) mediating BK_{Ca} channel activity.

### Regulation of Blood Pressure and Vascular Smooth Muscle Cell (VSMC) Contractility by Caᵥ1.2, Ryanodine Receptor (RyR) and BK_{Ca} Channels

Arterial blood pressure is determined by several factors, including vascular tone, which represents the contractile activity of smooth muscle within the walls of resistance vessels. The contractile state of smooth muscle is organized through the interplay of vasoconstrictor and vasodilatory neurohormones and by blood pressure itself (the Bayliss effect; constriction of the vessel after an increase in transmural pressure) (Bayliss, W. M., On the local reactions of the arterial wall to changes of internal pressure. J. Physiol., 1902. 28:220-23; Nelson, M. T., Bayliss, myogenic tone and volume-regulated chloride channels in arterial smooth muscle. J. Physiol., 1998. 507 (Pt 3):629; Nelson, et al., Noradrenaline contracts arteries by activating voltage-dependent calcium channels. Nature, 1988. 336(6197):382-5). The autoregulatory Bayliss effect is based upon graded membrane depolarization in response to pressure, which activates voltage dependent Ca²⁺ channels, causing vasoconstriction. Vascular smooth muscle contraction is triggered by Ca²⁺/calmodulin dependent phosphorylation of the regulatory myosin light chain. Increased intracellular Ca²⁺ is mediated by Ca²⁺ influx through Caᵥ1.2 and Ca²⁺ release from intracellular stores, mainly through the Inositol 1,4,5-Triphosphate Receptor (IP3R) (Davis, et al., Signaling mechanisms underlying the vascular myogenic response. Physiol. Rev., 1999. 79(2):387-423). Caᵥ1.2 was recently reported to play a critical role in regulating smooth muscle contraction/blood pressure regulation, as an inducible smooth muscle specific Caᵥ1.2 knockout demonstrated abnormal autoregulation and maintenance of vascular tone in response to depolarization and pressure (Moosmang, et al., Dominant role of smooth muscle L-type calcium channel Caᵥ1.2 for blood pressure regulation. Embo. J., 2003. 22(22):6027-34). In vascular smooth muscle, the dynamic range of intracellular calcium concentrations, [Ca²⁺]ᵢ, is narrow, ranging from ˜100 nM when the artery is maximally dilated to 350 nM when arteries are maximally constricted (Knot, et al., Regulation of arterial diameter and wall [Ca²⁺] in cerebral arteries of rat by membrane potential and intravascular pressure. J. Physiol, 1998. 508:199-209).

Spontaneous transient outward currents (STOC) were first described in smooth muscle by Bolton and coworkers (Benham, et al., Spontaneous transient outward currents in single visceral and vascular smooth muscle cells of the rabbit. J Physiol, 1986. 381:385-406; Bolton, et al., Spontaneous transient outward currents in smooth muscle cells. Cell Calcium, 1996. 20(2):141-52) and have been shown in a diverse group of vascular and non-vascular smooth muscle (Hisada, et al., Properties of membrane currents in isolated smooth muscle cells from guinea-pig trachea. Pflugers Arch., 1990. 416(1-2):151-61; Ohya, et al., Cellular calcium regulates outward currents in rabbit intestinal smooth muscle cell. Am. J. Physiol, 1987. 252(4 Pt I):C401-10; Saunders, et al., Spontaneous transient outward currents and Ca⁺⁺-activated K⁺ channels in swine tracheal smooth muscle cells. J. Pharmacol. Exp. Ther., 1991. 257(3): 1114-20; Nelson, et al., Relaxation of arterial smooth muscle by calcium sparks. Science, 1995. 270(5236):633-637; Nelson, et al., Physiological roles and properties of potassium channels in arterial smooth muscle. Am. J. Physiol, 1995. 268 (Cell Physiol. 37):C799-C822; Hume, et al., Macroscopic K⁺ currents in single smooth muscle cells of the rabbit portal vein. J. Physiol, 1989. 413:49-73; Jaggar, et al., Ca²⁺ channels, ryanodine receptors and Ca²⁺-activated K⁺ channels: a functional unit for regulating arterial tone. Acta Physiol. Scand., 1998. 164(4):577-87; Porter, et al., Frequency modulation of Ca²⁺ sparks is involved in regulation of arterial diameter by cyclic nucleotides. Am. J. Physiol, 1998. 274(5 Pt I):C1346-55). Each transient outward current represents the activation of 10-100 BK_{Ca} channels (Porter, et al., Frequency modulation of Ca²⁺ sparks is involved in regulation of arterial diameter by cyclic nucleotides. Am. J. Physiol, 1998. 274(5 Pt I):C1346-55). Nelson and colleagues obtained the first evidence of Ca²⁺ sparks in smooth muscle (Nelson, et al., Relaxation of arterial smooth muscle by calcium sparks. Science, 1995. 270(5236):633-637) and similar findings have been shown in numerous smooth muscle cells derived from arteries, portal vein, urinary bladder, gastrointestinal tract, airway and gallbladder (Mironneau, et al., Ca²⁺ sparks and Ca²⁺ waves activate different Ca²⁺-dependent ion channels in single myocytes from rat portal vein. Cell Calcium, 1996. 20(2): 153-60; Gordienko, et al., Crosstalk between ryanodine receptors and IP3Rs as a factor shaping spontaneous Ca²⁺-release events in rabbit portal vein myocytes. J. Physiol., 2002. 542(Pt 3):743-62; Herrera, et al., Voltage dependence of the coupling of Ca²⁺ sparks to BK_{Ca} channels in urinary bladder smooth muscle. Am. J. Physiol. Cell Physiol, 2001. 280(3):C481-90; Ji, et al., Stretch-induced calcium release in smooth muscle. J. Gen. Physiol, 2002. 119(6):533-44; Ji, et al., RYR2 proteins contribute to the formation of Ca²⁺ sparks in smooth muscle. J. Gen. Physiol, 2004. 123(4):377-86; Gordienko, et al., Variability in spontaneous subcellular calcium release in guinea-pig ileum smooth muscle cells. J. Physiol, 1998. 507 (Pt 3):707-20; Kirber, et al., Relationship of Ca²⁺ sparks to STOCs studied with 2D and 3D imaging in feline oesophageal smooth muscle cells. J. Physiol, 2001. 531(Pt 2):315-27). Ca²⁺ sparks are transient local increases in intracellular Ca²⁺ that occur through the coordinated opening of a group of RyR located on the SR (Nelson, et al., Relaxation of arterial smooth muscle by calcium sparks. Science, 1995. 270(5236):633-637). In cerebral artery myocytes, Ca²⁺ sparks lead to activation of the BK_{Ca} channel, thus providing an important feedback role in the regulation of pressure-induced constriction (Nelson, et al., Relaxation of arterial smooth muscle by calcium sparks. Science, 1995. 270(5236):633-637). Vasodilators may act, in part, through increasing the frequency of Ca²⁺ sparks.

All three RyR isoforms have been reported in smooth muscle (Marks, et al., Molecular cloning and characterization of the ryanodine receptor/junctional channel complex cDNA from skeletal muscle sarcoplasmic reticulum. Proc. Natl Acad. Sci., 1989. 86:8683-8687; Hakamata, et al., Primary Structure and distribution of a novel ryanodine receptor/calcium release channel from rabbit brain. FEBS, 1992. 312:229-235; Ledbetter, et al., Tissue distribution of ryanodine receptor isoforms and alleles determined by reverse transcription polymerase chain reaction. Journal of Biological Chemistry, 1994. 269(50):31544-51) although the relative proportion of each isoform varies between tissues (Xu, et al., Evidence for a Ca²⁺-gated ryanodine-sensitive Ca²⁺ release channel in visceral smooth muscle. Proc. Natl. Acad. Sci. USA, 1994. 91(8):3294-8). The physiologic role of each of the isoforms of the RyR is lacking. The respective roles of RyR2 and RyR1 in smooth muscle have been incompletely elucidated (Takeshima, et al., Excitation-contraction uncoupling and muscular degeneration in mice lacking functional skeletal muscle ryanodine-receptor gene. Nature, 1994. 369(6481):556-9; Takeshima, et al., Ca²⁺-induced Ca²⁺ release in myocytes from dyspedic mice lacking the type-1 ryanodine receptor. Embo. J., 1995. 14(13):2999-3006), in part, because RyR2 null mice are lethal (Takeshima, et al., Embryonic lethality and abnormal cardiac myocytes in mice lacking ryanodine receptor type 2. Embo. J., 1998. 17(12):3309-16). In rat portal vein myocytes, antisense oligonucleotides targeting each of the RyR isoforms demonstrated that both RyR1 and RyR2 are required for myocytes to respond to membrane depolarization with Ca²⁺ sparks and global increase in intracellular Ca²⁺ (Coussin, et al., Requirement of ryanodine receptor subtypes 1 and 2 for Ca²⁺-induced Ca²⁺ release in vascular myocytes J. Biol. Chem., 2000. 275(13):9596-603).

The RyR(Ca²⁺ spark)-BK_{Ca} channel complex can be viewed as a mechanism to limit smooth muscle contraction. Ca²⁺ spark frequency is increased when intravascular pressure is elevated from 10 to 60 mm Hg in rat cerebral arteries (Jaggar, J. H., Intravascular pressure regulates local and global Ca²⁺ signaling in cerebral artery smooth muscle cells. Am. J. Physiol. Cell Physiol., 2001. 281(2):C439-48). Inhibition of RyR or BKCa channels has been demonstrated to lead to pressure-induced cerebral artery constriction (Gollasch, et al., Ontogeny of local sarcoplasmic reticulum Ca²⁺ signals in cerebral arteries: Ca²⁺ sparks as elementary physiological events [published erratum appears in Circ. Res. 1999 Jan. 8-22; 84(1):125]. Circ. Res., 1998. 83(11):1104-14; Knot, et al., Ryanodine receptors regulate arterial diameter and wall Ca²⁺ in cerebral arteries of rat via Ca²⁺-dependent K⁺ channels. J. Physiol. (Lond), 1998. 508(Pt 1):211-21). BK_{Ca} channel from VSMC derived from β1 subunit knockout animals demonstrated ˜100-fold lower probability of opening and Ca²⁺ spark induced BK_{Ca} channel current was significantly reduced and greater than ⅓ of sparks failed to elicit a BK_{Ca} channel activation (Brenner, et al., Vasoregulation by the β1 subunit of the calcium-activated potassium channel. Nature, 2000. 407:870-876). Mean arterial pressure was elevated in the β1 subunit null animals, leading to left ventricular hypertrophy and hypertension (Id.). Thus, the ability of the BK_{Ca} channel to sense the Ca²⁺ sparks was impaired by the loss of the β1 subunit. In contrast, a gain of function mutation of β1 (G352A) was associated with a low prevalence of moderate and severe diastolic hypertension (Fernandez-Fernandez, et al., Gain-of-function mutation in the KCNMBI potassium channel subunit is associated with low prevalence of diastolic hypertension. J. Clin. Invest, 2004. 113(7): 1032-9). BK_{Ca}-β1_{E65K} channels showed increased Ca²⁺ sensitivity (Id.). Activation of the PKA and PKG signal transduction pathways leads to 2-3 fold increases in both Ca²⁺ spark and BK_{Ca} channel activity (Porter, et al., Frequency modulation of Ca²⁺ sparks is involved in regulation of arterial diameter by cyclic nucleotides. Am. J. Physiol, 1998. 274(5 Pt I):C1346-55; Wellman, et al., Role of phospholamban in the modulation of arterial Ca²⁺ sparks and Ca²⁺-activated K⁺ channels by cAMP. Am. J. Physiol. Cell Physiol, 2001. 281(3):C1029-37). Ryanodine reduced dilation to forskolin by 80%, consistent with the importance of Ca²⁺ sparks and a potential regulatory role of PKA. However, in arterial smooth muscle derived from phospholamban null mice, forskolin had little effect compared to the ˜2 fold increase in Ca²⁺ spark frequency in wild type animals (Wellman, et al., Role of phospholamban in the modulation of arterial Ca²⁺ sparks and Ca²⁺-activated K⁺ channels by cAMP. Am. J, Physiol. Cell Physiol, 2001. 281(3):C1029-37).

### Modulators of BK_{Ca} Channel Activity

Acute pharmacological inhibition of BK channels has been shown to increase ASM baseline contractility, enhance cholinergic-mediated contraction and prevent isoproterenol-mediated relaxation of tracheal rings (Jones et al., Selective inhibition of relaxation of guinea-pig trachea by charybdotoxin, a potent Ca(++)-activated K+ channel inhibitor. J Pharmacol Exp Ther 255:697-706 (1990); Murray et al., Receptor-activated calcium influx in human airway smooth muscle cells. J Physiol 435:123-144 (1991); Corompt et al., Inhibitory effects of large Ca²⁺-activated K⁺ channel blockers on beta-adrenergic- and NO-donor-mediated relaxations of human and guinea-pig airway smooth muscles. Naunyn Schmiedebergs Arch Pharmacol 357:77-86 (1998); Jones et al., Interaction of iberiotoxin with betaadrenoceptor agonists and sodium nitroprusside on guinea pig trachea. J Appl Physiol 74:1879-1884 (1993)).

Supporting the important role of BK channels in ASM contractility are several recent findings. First, a report (Sausbier et al., Reduced rather than enhanced cholinergic airway constriction in mice with ablation of the large conductance Ca²⁺-activated K⁺ channel. FASEB J 21:812-822 (2007)) demonstrated that the membrane potential of BK a null mice tracheal SMC are ˜10 mV less negative than the membrane potential of cells from WT mice. However, BK α null animals had a paradoxical phenotype of reduced sensitivity of the airways toward bronchoconstrictors and an enhanced sensitivity toward bronchodilators. Both effects were the result of compensatory mechanisms involving the amplification of cGMP signaling proteins, suggesting that BK channels play such an important role in airway physiology that long-term adaptation mechanisms compensate for the loss of functional channels (Id.). Second, another report (Semenov et al., BK channel beta1-subunit regulation of calcium handling and constriction in tracheal smooth muscle. Am J Physiol Lung Cell Mol Physiol 291:L802-810 (2006)) demonstrated that increased resting [Ca²⁺]ᵢ and increased sustained component of Ca²⁺ influx after cholinergic stimulation in tracheal SMC isolated from BK β1 null mice compared to WT mice. Third, in an African-American asthmatic population, a BK β1 subunit polymorphism (R140W) is associated with a clinically significant decline (−13%) in FEV1 in males, but not females (Seibold et al., An African-specific functional polymorphism in KCNMB1 shows sex specific association with asthma severity. Hum Mol Genet 17:2681-2690 (2008)). R140W is in the extracellular loop of β1 and suppresses β1 enhancement of BK sensitivity to Ca²⁺. It is apparent that the extracellular loop of β1 plays an important role in modulating α, since a different polymorphism in β1, E65K, is associated with a decreased incidence of diastolic hypertension and heart disease due to a gain-of-function (Fernandez-Fernandez et at, Gain-of-function mutation in the KCNMB1 potassium channel subunit is associated with low prevalence of diastolic hypertension. J Clin Invest, 113(7): p. 1032-9 (2004)). Electrophysiology studies of α and R140W mutant β1 subunits demonstrated significantly reduced channel openings.

Pharmacologic approaches to activate BK_{Ca} channels represent a new/emerging strategy to control membrane excitability. Despite the increasing number of natural and synthetic BK_{Ca} channel openers, relatively little is known about the interaction sites and mechanism of action. Moreover, many of the compounds are relatively weak, with nonspecific activity towards BK_{Ca} channels (Ohwada, et al., Dehydroabietic acid derivatives as a novel scaffold for large-conductance calcium-activated K⁺ channel openers. Bioorg. Med. Chem. Lett., 2003. 13(22):3971-4). Small natural or synthetic products could have effectiveness in diseases mediated through muscular and neuronal hyperexcitability such as asthma, urinary incontinence/bladder spasm, gastroenteric hypermotility, hypertension, coronary spasm, psychoses, convulsion and anxiety (Calderone, V., Large-conductance, Ca²⁺-activated K⁺ channels: function, pharmacology and drugs. Curr. Med. Chem., 2002. 9(14):1385-95; Pelaia, et al., Potential role of potassium channel openers in the treatment of asthma and chronic obstructive pulmonary disease. Life Sci, 2002. 70(9):977-90; Gribkoff, et al., The pharmacology and molecular biology of large-conductance calcium-activated (BK) potassium channels. Adv. Pharmacol, 1997. 37:319-48; Nardi, et at, Natural modulators of large-conductance calcium-activated potassium channels. Planta. Med., 2003. 69(10):885-92). Recent work has suggested a role for K⁺ channel activators for post-stroke neuroprotection, erectile dysfunction and cardiac diseases such as coronary artery vasospasm/hypertension (Nardi, et al., Natural modulators of large-conductance calcium-activated potassium channels. Planta. Med., 2003. 69(10):885-92; Gribkoff, et al., Targeting acute ischemic stroke with a calcium-sensitive opener of maxi-K potassium channels. Nat. Med., 2001. 7(4):471-7).

The synthetic benzimidazolone derivatives NS004 and NS1619 are the pioneer BK-activators (activate BK_{Ca} current at 10-30 μM in vascular and non-vascular smooth muscle) (Coghlan, et al., Recent developments in the biology and medicinal chemistry of potassium channel modulators: update from a decade of progress. J. Med. Chem. 2001. 44(11):1627-53) and have led to the design of several novel and heterogenous BK-openers (Olesen, et al., NS004-an activator of Ca²⁺-dependent K⁺ channels in cerebellar granule cells. Neuroreport, 1994. 5(8): 1001-4; Olesen, et al., Selective activation of Ca²⁺-dependent K⁺ channels by novel benzimidazolone. Eur. J. Pharmacol., 1994. 251(I):53-9). In addition to BK_{Ca} channel opening, NS-1619 inhibits Ca²⁺ and Cl⁻ channels (Gribkoff, et al., The pharmacology and molecular biology of large-conductance calcium-activated (BK) potassium channels. Adv. Pharmacol, 1997. 37:319-48), but has been reported to increase intracellular Ca²⁺ concentration (at 30 μM) in porcine coronary myocytes through the ryanodine receptor sensitive storage sites (Yamamura, et al., BK channel activation by NS-1619 is partially mediated by intracellular Ca²⁺ release in smooth muscle cells of porcine coronary artery. Br. J. Pharmacol, 2001. 132(4):828-34). NS1608 caused BK_{Ca} channel activation (minimum effective concentration 0.5 μM; maximum between 5-10 μM), but demonstrated a bell shaped concentration with an inhibitory effect at higher concentrations (50 μM) in porcine coronary artery cells (Hu, et al., Differential effects of the BK_{Ca} channel openers NS004 and NS1608 in porcine coronary arterial cells. Eur. J. Pharmacol, 1995. 294(1):357-60; Hu, et al., On the mechanism of the differential effects of NS004 and NS1608 in smooth muscle cells from guinea pig bladder. Eur. J. Pharmacol, 1996. 318:461-8). BMS-204352 (MaxiPost) has been evaluated in clinical trials for stroke therapy and a reduction in brain infarct size has been detected in rat stroke models (Gribkoff, et al., Targeting acute ischemic stroke with a calcium-sensitive opener of maxi-K potassium channels. Nat. Med, 2001. 7(4):471-7; Imaizumi, et al., Molecular basis of pimarane compounds as novel activators of large-conductance Ca²⁺-activated K⁺ channel alpha-subunit Mol. Pharmacol, 2002. 62(4):836-46). The effects of BMS-204352 were Ca²⁺ sensitive; at 50 nM intracellular Ca²⁺, the compound had almost no effect, whereas at higher intracellular Ca²⁺ concentrations, it produced progressively greater increases in current (Gribkoff, et al., Targeting acute ischemic stroke with a calcium-sensitive opener of maxi-K potassium channels. Nat. Med, 2001. 7(4):471-7). Three glycosylated triterpenes called dehydrosoyasaponin-I (DHS-I), soyasaponins I and III have been shown to activate BK_{Ca} channels. DHS-I has poor membrane permeability, but is probably metabolized to other active molecules that penetrate the cell. DHS-I increases channel activity when the α and β subunits are co-expressed (McManus, et al., An activator of calcium-dependent potassium channels isolated from a medicinal herb. Biochemistry, 1993. 32(24):6128-33; Giangiacomo, et al., Mechanism of maxi-K channel activation by dehydrosoyasaponin-I. J. Gen. Physiol, 1998. 112(4):485-501). Maxikdiol, a 1,5-dihydroxyisoprimane diterpenoid has limited membrane permeability, but can activate the channel (threshold-1 μM; significant effect-3-10 μM) when applied to the cytoplasmic side (Nardi, et al., Natural modulators of large-conductance calcium-activated potassium channels. Planta. Med., 2003. 69(10):885-92; Singh, et al., Maxikdiol: a novel dihydroxyisoprimane as an agonist of maxi-K channels. J. Chem. Soc. Perkin Trans., 1994. 1:3349-3352; Kaczorowski, et al., High-conductance calcium-activated potassium channels; structure, pharmacology, and function. J. Bioenerg. Biomembr., 1996. 28(3):255-67; Lawson, K., Potassium channel openers as potential therapeutic weapons in ion channel disease. Kidney Int., 2000. 57(3):838-45).

### Rottlerin

Rottlerin (mallotoxin), a natural product from Mallotus phillippinensis, is a 5,7-dihydroxy-2,2-dimethyl-6-(2,4,6-trihydroxy-3-methyl-5-acetylbenzyl)-8-cinnamoyl-I,2-chromene that has been frequently used as a protein kinase Cδ (PKCδ) inhibitor based upon an in vitro study demonstrating that the IC₅₀ for PKCδ and CaMK III were 3-6 μM compared to 30-100 μM for other PKC isozymes, protein kinase A (PKA) and casein kinase II (Gschwendt, et al., Rottlerin, a novel protein kinase inhibitor. Biochem. Biophys. Res. Commun., 1994. 199(I):93-8). Based upon rottlerin, a role for PKCδ in a variety of biological events including apoptosis, cell differentiation, mitogen activated protein kinase activation and other cell processes was described. Rottlerin inhibits an increase of histamine in BAL fluid from OVA-challenged animals compared to animals challenged with PBS (Cho et al., “Protein kinase Cδ functions downstream of Ca²⁺ mobilization in FcεRI signaling to degranulation in mast cells” J Allergy Clin Immunol, 114:1085-1092 (2004)).

More recent data suggest that rottlerin is ineffective in blocking PKCδ activity in vitro, but can uncouple mitochondria (10 μM) in intact cells and reduce ATP levels in a PKC independent fashion (Soltoff, S. P., Rottlerin is a mitochondrial uncoupler that decreases cellular ATP levels and indirectly blocks protein kinase Cδ tyrosine phosphorylation. J. Biol. Chem., 2001. 276(41):37986-92; see also Soltoff, S. P., Rottlerin: an inappropriate and ineffective inhibitor of PKCδ. Trends in Pharm. Sci. 28(9):453-458 (August 2007)), potentially sensitizing colon carcinoma cells to tumor necrosis factor-related apoptosis (Tillman, et al., Rottlerin sensitizes colon carcinoma cells to tumor necrosis factor-related apoptosis-inducing ligand-induced apoptosis via uncoupling of the mitochondria independent of protein kinase C. Cancer Res., 2003. 63(16):5118-25). In standard assays at 0.1 mM ATP (or even 0.01 mM), rottlerin (20 μM) had virtually no effect on PKCα or PKCδ activity in the presence of phosphatidylserine (PS) using either histone H1 or myelin basic protein as a substrate (Davies, et al., Specificity and mechanism of action of some commonly used protein kinase inhibitors. Biochem. J., 2000. 351(R I):95-105).

Rottlerin does inhibit several other kinases, including p38-regulated/activated protein kinase (PRAK) and mitogen-activated protein kinase with similar in vitro potencies as PKCδ. In addition, 20 μM rottlerin as been shown to substantially inhibit c-Jun N-terminal kinase 1 α1 (JNK1 α1, 51% inhibition), mitogen- and stress-activated protein kinase 1 (MSK-1, 62% inhibition), PKA (83% inhibition), 3-phosphoinositide-dependent protein kinase-1 (PDK-1, 64% inhibition), Akt (73% inhibition) and glycogen synthase kinase 3βGSK3β, 87% inhibition) (Soltoff, S. P. “Rottlerin: an inappropriate and ineffective inhibitor of PKCδ” Trends Pharmacol Sci 28:453-458 (2007); Davies et al., “Specificity and mechanism of action of some commonly used protein kinase inhibitors” Biochem J 351:95-105 (2000)). Rottlerin has also been reported to inhibit insulin-induced glucose uptake (IC₅₀=10 μM) in 3T3-L1 adipocytes (Kayali, et al., Rottlerin inhibits insulin-stimulated glucose transport in 3T3-L1 adipocytes by uncoupling mitochondrial oxidative phosphorylation. Endocrinology, 2002. 143(10):3834-96). Rottlerin has been reported to decrease the capacity for the glutamate-aspartate transporter (GLAST) subtype of the glutamate transporter (Susarla, et al., Rottlerin, an inhibitor of protein kinase Cδ (PKCδ), inhibits astrocytic glutamate transport activity and reduces GLAST immunoreactivity by a mechanism that appears to be PKCδ-independent J. Neurochem., 2003. 86(3):635-45).

Rottlerin also inhibits, in a dose-dependent manner, CD4⁺ and CD8⁺ human T lymphocyte proliferation in response to anti-CD3/anti-CD28 antibodies. The inhibition was associated with impaired CD25 expression, decreased IL-2 production and decreased mRNA expression of interferon γ, IL-10 and IL-13 activated T cells (Springael et al., “Rottlerin inhibits human T cell responses” Biochem Pharmacol 73:515-525 (2007)). Rottlerin blocked PMA-induced phosphorylation of Erk-1 and Erk-2 in Jurkat T cells and purified human CD4+ T cells from peripheral blood (Roose et al., “A diacylglycerol-protein kinase C-RasGRP1 pathway directs Ras activation upon antigen receptor stimulation of T cells” Mol Cell Biol 25:4426-4441 (2005)).

The history/development of rottlerin as a therapeutic compound is complex. Kamala, from which rottlerin may be purified, has been used in India for centuries as an antihelmintic. Kamala is collected from the capsules of Mallotus philippinensis, a tree grown in Abyssinia, Southern Arabia, Hindostan, the East India Islands, China, and Australia (Remington et al., ed, The Dispensatory of the United States of America, 1918). Kamala, when examined under the microscope, consists of garnet-red, semi-transparent, roundish, glandular hairs from 0.040 to 0.100 mm in diameter, and containing numerous red, club-shaped cells and admixed with minute stellate hairs, and the remains of stalks and leaves, the latter of which are easily removed by careful sifting. (Id.) The most important constituent of Kalama is a dark brownish red resin (about 80%) composed chiefly of a crystalline chemical, rottlerin and a yellowish crystalline isomer, isorottlerin. (Gujral, et al., Oral contraceptives. Part II. Antifertility effect of Mallotus philippinensis Mueller-argoviensis. Indian J. Med. Res., 1960. 48:52-8). Thomas Anderson of Glasgow found that kamala consists of 78.19% resinous coloring matter, 7.34% albumin, 7.14% cellulose and the like, a trace of volatile oil and volatile coloring matters, 3.84% ashes, and 3.49% water. (Remington et al., ed, The Dispensatory of the United States of America, 1918) The amount of earthy impurities, chiefly sand, in commercial kamala, varies greatly, sometimes amounting to fifty or even sixty per cent. (Id.)

Kamala is violently purgative in full doses, occasionally causing nausea but seldom vomiting (Gujral, et al., Oral contraceptives. Part II. Antifertility effect of Mallotus philippinensis Mueller-argoviensis. Indian J. Med. Res., 1960. 48:52-8). In 1910, Semper reported that kamala caused a paralyzing effect on motor nerves and muscle (Id.), which based on the inventors' data is likely due to its effects on the BK_{Ca} channel. Kamala has been used in India against the tapeworm and is given to patients (3.9-11.6 grams) suspended in water, mucilage or syrup. The worm is usually expelled at the third or fourth stool. As an external remedy, kamala is used by the people of India for various afflictions of the skin, particularly scabies.

Rottlerin appears to have been isolated ˜150 years ago (1855) by Anderson (Anderson, A., Kamala resin-rottlerin. Edin. New Phil. Jour., 1855. 1:296-300), by allowing a concentrated ethereal solution of kamala to stand for two days, draining and pressing the granular crystals in bibulous paper and purifying them from the adhering resin. Perkin and Perkin confirmed the substance and called it mallotoxin (Gujral, et al., Oral contraceptives. Part II. Antifertility effect of Mallotus philippinensis Mueller-argoviensis. Indian J. Med. Res., 1960. 48:52/−8). Such isolation methods are incorporated by reference as if recited in full herein. Pure rottlerin has the chemical composition C₃₃H₃₀O₉ and has been reported to exist in both the keto and enol forms.

Rottlerin has been given to animals; it has been shown to reduce the fertility rate of rats and guinea pigs (dose of purified rottlerin=10-20 mg/kg/day×6 days) (Id.). Injection of rottlerin (5 μM) into the cistera magna in a canine subarachnoid hemorrhage model inhibited the initial phase of cerebral vasospasm, which was attributed to its effects on PKCδ (Nishizawa, et al., Attenuation of canine cerebral vasospasm after subarachnoid hemorrhage by protein kinase C inhibitors despite augmented phosphorylation of myosin light chain. J. Vase. Res., 2003. 40(2):169-78). It is conceivable that some of the beneficial effects may have been secondary to the effects on BK_{Ca} channel. The RTECS database (AM6913800) indicates no information regarding LD₅₀/LC₅₀ for acute/chronic toxicity.

In view of the foregoing, new and improved methods and compositions for modulating ASM would be desirable. The present invention is directed to achieving these and other objectives.

### SUMMARY OF THE INVENTION

Disclosed herein is that rottlerin and derivatives thereof are potent activators of the BK channel and that asthma, hypertension, and related disorders can be treated or prevented via regulation of the BK channel using rottlerin. Accordingly, the present invention provides compositions and methods for regulating the BK channel using rottlerin and derivatives thereof. Pharmaceutical compositions and methods for treating, preventing, or ameliorating the effects of asthma are also provided.

One embodiment of the present invention is a method of treating or ameliorating the effects of a disease characterized by altered smooth muscle contractility. This method comprises administering to a patient suffering from such a disease an effective amount of a large-conductance Ca²⁺-activated K⁺ (BK) channel modulator.

Another embodiment of the present invention is a method of treating or ameliorating the effects of asthma. This method comprises administering to a patient suffering from asthma an effective amount of a BK channel modulator.

A further embodiment of the present invention is a method for decreasing airway constriction and/or airway resistance in a patient without increasing the heart rate of the patient. This method comprises administering to the patient an effective amount of a BK channel modulator or a pharmaceutical composition comprising a pharmaceutically acceptable carrier and a BK channel modulator.

Yet another embodiment of the present invention is a method for modulating inflammation in a lung of a patient. This method comprises administering to a patient an effective of amount of a BK channel modulator, or a pharmaceutical composition comprising a pharmaceutically acceptable carrier and a BK channel modulator, which amount is sufficient to modulate the inflammation.

An additional embodiment of the present invention is a pharmaceutical composition for treating or ameliorating the effects of a disease characterized by altered smooth muscle contractility. This pharmaceutical composition comprises a pharmaceutically acceptable carrier and a BK channel modulator.

Another embodiment of the present invention is a pharmaceutical composition for treating, preventing, or ameliorating the effects of asthma. This pharmaceutical composition comprises a pharmaceutically acceptable carrier and a BK channel modulator.

In another aspect of the present invention, the above-described compounds and pharmaceutical compositions can be used to regulate membrane excitability both in vitro and in vivo. In one example, the compounds and pharmaceutical compositions of the present invention can be used to treat or prevent a hyperexcitability disorder.

In an embodiment of the invention, the hyperexcitability disorder is asthma. In another embodiment of the invention, the hyperexcitability disorder is hypertension. In other embodiments of the present invention, the hyperexcitability disorder includes, but is not necessarily limited to urinary incontinence, gastroenteric hypermotility, coronary spasm, psychoses, convulsion and anxiety. In another embodiment, the compounds and pharmaceutical compositions of the present invention are used in treating or preventing erectile dysfunction. In yet other embodiments, the compounds and pharmaceutical compositions of the present invention are used in treating or preventing coronary artery vasospasm and hypertension. In another embodiment, the compounds and pharmaceutical compositions of the present invention are used in treating or preventing neurologic dysfunction. In an additional embodiment, the compounds and pharmaceutical compositions of the present invention are used in post-stroke neuroprotection.

The present invention also provides methods for treating or preventing a hyperexcitability disorder in a subject, comprising administering to the subject a therapeutically effective amount of the pharmaceutical composition of the invention. In an embodiment of the invention, the hyperexcitability disorder includes, but is not necessarily limited to, asthma, urinary incontinence, gastroenteric hypermotility, hypertension, coronary spasm, psychoses, convulsion and anxiety.

The present invention also provides methods for treating or preventing erectile dysfunction in a subject by administering to the subject a therapeutically effective amount of a pharmaceutical composition of the invention. Additionally, the present invention also provides methods for treating or preventing a coronary artery vasospasm in a subject, comprising administering to the subject a therapeutically effective amount of a pharmaceutical composition of the present invention.

The invention additionally provides methods for treating or preventing hypertension in a subject, comprising administering to the subject a therapeutically effective amount of a pharmaceutical composition of the present invention.

The present invention further encompasses methods for treating or preventing a neurologic dysfunction in a subject, comprising administering to the subject a therapeutically effective amount of a pharmaceutical composition of the present invention. The present invention also provides methods for post-stroke neuroprotection in a subject by administering a therapeutically effective amount of a pharmaceutical composition of the present invention.

The present invention further provides kits for use in treating or preventing hyperexcitability disorders in a subject comprising a therapeutically effective amount of a pharmaceutical composition of the present invention, optionally, in combination with a pharmaceutically acceptable carrier. In an embodiment, the hyperexcitability disorder includes, but is not necessarily limited to, asthma, urinary incontinence, gastroenteric hypermotility, hypertension, coronary spasm, psychoses, convulsion and anxiety.

The present invention also provides kits for use in treating or preventing erectile dysfunction, coronary artery vasospasm, hypertension or neurologic dysfunction in a subject, comprising administering a therapeutically effective amount of a pharmaceutical composition of the present invention.

Finally, the present invention also provides kits for use in post-stroke neuroprotection in a subject, comprising a therapeutically effective amount of a pharmaceutical composition of the present invention.

### BRIEF DESCRIPTION OF THE DRAWINGS

FIG. 1 depicts a representation of PKA and β1-subunit regulation of BK_{Ca} channel. FIG. 1A illustrates that signaling through the BK_{Ca}-associated β2AR leads to cAMP generation, PKA phosphorylation of S872 (mSlo), and increased channel activity. Increased channel activity may be due to Gα association. FIG. 1B illustrates that β1 subunit modification of BK_{Ca} channel leads to activation.

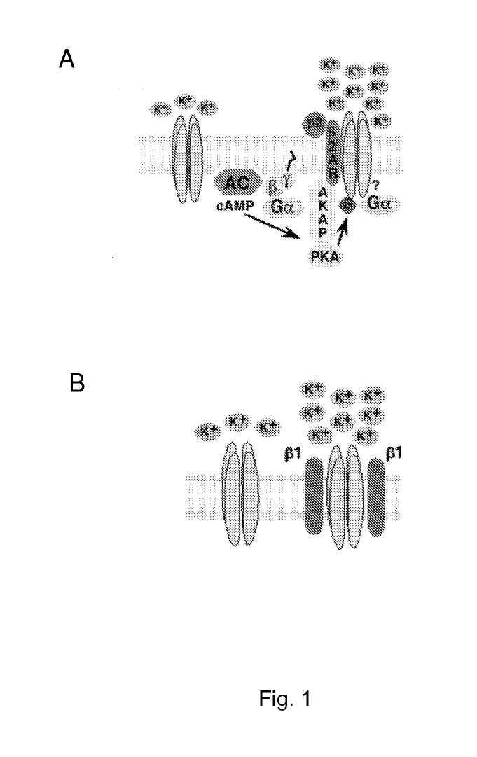

FIG. 2 shows a schematic of the structure of an α subunit of the BK_{Ca} channel. The α subunit is the pore forming subunit; the tetrameric channel is formed by four α subunits. Seven transmembrane domains are shown: S0-S6. The pore is between S5 and S6. The channel has a unique C-terminus, with four additional, non-transmembrane hydrophobic regions (S7-S10). The Ca²⁺ regulatory domains are indicated; the Ca²⁺ bowl, M513 and D362/D367 form independent high affinity Ca²⁺ sensors. The RCK1 and RCK2 domains are indicated. Adapted from (Magleby, K. L., Gating mechanism of BK (Slo1) channels: so near, yet so far. J. Gen. Physiol, 2003. 121(2):81-96).

FIG. 3 is a schematic representation of the role of RyR in regulation of smooth muscle cell (SMC) constriction and dilation. Local Ca²⁺ release (sparks) from RyR activate BK_{Ca}, whose outward current (spontaneous transient outward currents; STOC) hyperpolarize the membrane and inhibit voltage gated Ca²⁺ channels (Caᵥ1.2). Agents that increase cAMP in vascular SMC cause vasodilatation. PKA has a direct effect on BK_{Ca}, but also increases spark activity, potentially by increased phosphorylation of the voltage gated Ca²⁺ channel, RyR, and phospholamban (adapted from (Porter, et al., Frequency modulation of Ca²⁺ sparks is involved in regulation of arterial diameter by cyclic nucleotides. Am. J. Physiol., 1998. 274 (Cell Physiol. 43):C1346-C1355)). The presence of the IP3R on the sarcoplasmic reticulum (SR) is not shown. The β2AR is shown associated with BK_{Ca} and Caᵥ1.2, whereas β1 adrenergic receptor (AR) does not associate with either channel. The regulation of BK_{Ca} and RyR by other kinases is not shown.

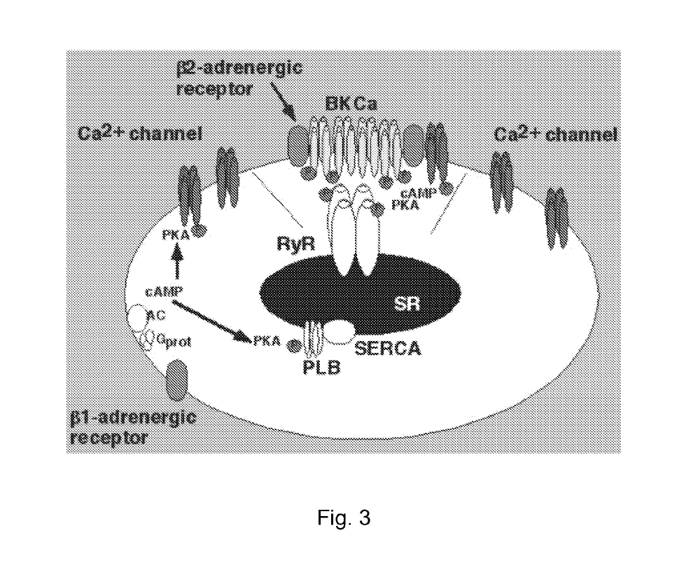

FIG. 4 shows electrophysiologic characterization of rottlerin. FIGS. 4A and 4B show representative current traces from whole cell patches with 5 mM EGTA in patch pipette. Rottlerin (0.5 μM) was applied to the extracellular side through local perfusion. Voltage steps are shown at the right of each tracing; note the different maximum voltage steps +200 mV (upper) vs. +120 mV (lower). Rottlerin significantly prolonged tail currents indicative of slowing of deactivation. Tail currents are in the opposite direction due to the final voltage step (+60 upper, −60 mV left). FIG. 4C G-V curves were constructed for indicated conditions utilizing tail analysis (Xia, et al., Multiple regulatory sites in large-conductance calcium-activated potassium channels. Nature, 2002. 418(6900):880-4). Solid lines were fitted with Boltzmann function. Exposure to rottlerin shifted the G-V curve in the hyperpolarizing direction (indicative of activation). Representative of more than 30 similar experiments with rat and murine (pictured) slo. FIGS. 4D and 4E show experiments studying stable mslo HEK293 cells in whole cell configuration with a pipette solution containing 5 mM EGTA (˜0 free Ca²⁺). Local perfusion of rottlerin (extracellular; 0.5 μM) activated channel. Exposure of cell to BK_{Ca} channel blocker tetraethylammonium (TEA) (5 mM) reversibly inhibited rottlerin induced BK_{Ca} activation.

FIG. 5 shows that intracellular exposure of rottlerin through dialysis for an extended period has minimal effect on BK_{Ca} channel activity. FIG. 5A shows representative current traces made 1 minute and 25 minutes after establishing whole cell voltage patch clamp, dialyzed with 5 mM EGTA (˜0 cytosolic Ca²⁺) and 20 μM rottlerin in a patch pipette. Over 25 minutes, intracellular dialysis of rottlerin had minimal effect on BK_{Ca} current. After 25 minutes, the cell was exposed to rottlerin (0.5 μM) via local perfusion, which significantly increased channel activity as shown in the diary plot (FIG. 5B) and the G-V curve (FIG. 5C). Current in the diary plot in FIG. 5B represents maximal current in ramp protocol at +70 mV. Holding potential of ramp was −20 mV, with ramp from −30 mV to +180 mV over 500 ms. The G-V curve in FIG. 5C was generated from the tail analysis as described in FIG. 4. Exposure of rottlerin through cytoplasmic administration had minimal effect on the channel; only exposure through the extracellular space caused activation, suggesting that the compound requires access to the privileged space only accessible extracellularly.

FIG. 6 shows single channel recordings of a BK_{Ca} channel. FIG. 6A shows representative outside-out single channel traces demonstrating activation of a BK_{Ca} channel from local perfusion of rottlerin (0.5 μM) to the extracellular side. Amplitude histograms are shown on the right. Rottlerin does not significantly change the channel conductance. Ca²⁺ was maintained at 0 (virtual; actual <20 nM) through dialysis using a patch pipette. Because cellular compartments are excluded from patch and outside-out patch perfused locally with 0 Ca²⁺, activation is Ca²⁺-independent. Channel activation can be inhibited by iberiotoxin or TEA (not shown). FIG. 6B shows representative inside-out single channel traces demonstrating activation of BK_{Ca} with local perfusion of rottlerin (0.5 μM) in the presence of 0 Ca²⁺. Amplitude histograms are shown on the right.

FIG. 7 shows that rottlerin activates BK_{Ca} channel in Human Embryonic Kidney (HEK) and VSMC cells. (A) Comparison of the effects of NS-1619 and rottlerin. Time course of whole cell voltage clamp experiment in a stably transfected mSlo HEK293 cell demonstrating current at +60 mV. Current was monitored with a ramp protocol; holding potential −60 mV, with ramp from −80 mV to +150 mV over 500 ms. Extracellular application of NS-1619 (10 μM) increased current as previously described (Olesen, et al., Selective activation of Ca²⁺-dependent K⁺ channels by novel benzimidazolone. Eur. J. Pharmacol., 1994. 251(1):53-9). After stabilization of the current, rottlerin (0.5 μM) was applied to the cell by local perfusion. Rottlerin shifted the V_{0.5} by ˜100 mV after 5 minutes. Analysis was performed using tail analysis with normalization as previously described (Xia, et al., Multiple regulatory sites in large-conductance calcium-activated potassium channels. Nature, 2002. 418(6900):880-4). FIG. 7B shows a study using HEK cells co-expressing α and β1 subunits in whole cell, configuration with 5 mM EGTA (intra-pipette). Rottlerin (0.5 μM) significantly shifted the I-V curve to the left, similar to results in HEK cells expressing only the α subunit (see FIG. 5). FIG. 7C shows a study using human VSMC in outside-out configuration, utilizing single channel recordings, recorded at +60 mV. Exposure of the same patch to rottlerin (0.5 μM) by local perfusion significantly increased Po and open dwell time. Identification of the BK_{Ca} channel was determined by conductance and inhibition by iberiotoxin and TEA. FIG. 7D shows that rottlerin inhibits phenylephrine (PE) induced modulation of VSMC tone. Murine femoral arterial rings were isolated and placed in a wire myograph. PE induced constriction of the vessel was significantly inhibited by rottlerin (0.5 μM). Rottlerin's effects were inhibited by TEA. The figures shown are representative of 4 similar experiments. Error bars are standard error of the mean (SEM). Asterisk (*) indicates p<0.001.

FIG. 8 shows the results of tracheal constriction studies. FIG. 8A shows the cumulative dose-response curves of WT tracheal rings to isoproterenol in the presence of a β1-AR antagonist (CGP 20712A; 100 nM) or a β2-AR antagonist (ICI 118551; 100 nM) or vehicle (DMSO). (n=4 each). Asterisk (*) indicates P<0.05 versus vehicle (DMSO). FIG. 8B shows that BK channels are required for β-agonist mediated tracheal ring relaxation. Cumulative dose-response curves of WT tracheal rings to isoproterenol in the absence and presence of 100 nM iberiotoxin (IbTX), a specific BK channel inhibitor. Values in graphs are means±standard deviation.

FIG. 9A shows the timeline of rottlerin administration in an ovalbumin (OVA) induced acute asthma model. FIG. 9B shows airway responsiveness in mice following OVA challenge and rottlerin administration. n=4 per group. Asterisk (*) indicates P<0.05 for OVA compared to OVA+Rottlerin.

FIG. 10 shows that rottlerin reduces the inflammatory response in asthma. FIG. 10A shows differential cell count of bronchoalveolar lavage fluid (BALF) from lungs of mice. Data are expressed as mean+SEM. (n=17 per group). **p<0.01 (OVA+PBS compared to PBS+PBS); *p<0.05 (OVA+PBS compared to OVA+rottlerin); #p<0.05, ##p<0.01 (OVA+rottlerin compared to PBS+Rottlerin). FIG. 10B shows the levels of OVA specific IgE as determined by ELISA. Data are expressed as mean+S.D. (n=17 per group). **p<0.01 (OVA+PBS compared to PBS+PBS); ##p<0.01 OVA+rottlerin compared to PBS+rottlerin. FIG. 10C shows the levels of cytokines in BALF. BALF from 3-week, HDM-sensitized mice treated with PBS or rottlerin analyzed for Th2 cytokines IL-4, IL-5 and IL-13 (n=17 per group). *p<0.05 OVA+PBS compared to OVA+rottlerin; ##p<0.01 OVA+PBS/rottlerin compared to PBS+PBS/rottlerin.

FIG. 11 shows that rottlerin activates BK channels and hyperpolarizes the membrane potential. FIG. 11 shows G-V curves, which were generated from tail current analysis for control conditions (triangle) and after rottlerin (0.5 μM) exposure (squares) utilizing Boltzmann function. In this experiment, murine tracheal smooth muscle cells were acutely isolated and whole cell patch clamped as previously described (Zakharov, S. et al., (2005) J Biol Chem 280, 30882-30887). FIG. 11B shows representative trace of membrane potential measurement before and after application of rottlerin (1 μM) and paxilline (10 μM). FIG. 11D shows a graph of membrane potential changes in ASM following rottlerin and paxilline administration. FIG. 11C shows cumulative dose-response curves of WT tracheal rings to ISO (1 nM to 100 μM) in the presence of PBS, rottlerin or rottlerin+iberiotoxin (n=5 per group). Values are means+S.D. *p<0.05 for rottlerin compared to rottlerin+iberiotoxin.

FIG. 12 shows that rottlerin activation of BK channels is not dependent on cellular signaling pathways. FIG. 12A shows representative outside-out patch single channel traces recorded at +30 mV of recombinant BK (mSlo1) from control conditions and after bath application of rottlerin (0.5 μM). Activation was found in 100% of experiments (n=18). FIG. 12B shows representative outside-out patch single channel traces of cultured human VSMC, recorded at +60 mV in ˜20 nM Ca²⁺, from control conditions and after bath application of rottlerin (0.5 μM).

FIG. 13 shows that rottlerin enhances the isoproterenol-induced relaxation of tracheal rings on a myograph. Cumulative dose-response curves of WT tracheal rings of PBS- & OVA-sensitized groups to isoproterenol with or without rottlerin (n=5 per group) are shown. Values are means±S.D. Asterisk (*) indicates P<0.05 for OVA compared to OVA+Rottlerin.

FIG. 14 shows that rottlerin reduces airway resistance in an Ova-sensitized asthma model. FIG. 14A shows airway responsiveness in mice following OVA challenge and rottlerin administration. The number of mice in each group is as follows: PBS/PBS n=4, PBS/Rottlerin n=5, Ova/PBS n=6, Ova/Rottlerin n=5. Triple asterisks (***) indicate p<0.001 and asterisk (*) indicates p<0.05 for OVA/PBS compared to OVA/Rottlerin. FIG. 14B shows airway responsiveness in mice following OVA challenge and rottlerin administration in response to isoproterenol (n=4 per group). Asterisk (*) indicates P<0.05 for OVA compared to OVA+Rottlerin.

FIG. 15 shows that rottlerin activates airway smooth muscle BK channels. The figure shown is representative of 3 similar experiments in which tracheal smooth muscle cells were acutely isolated from mice and exposed to rottlerin (2 μM), and the membrane potential was determined using perforated patch.

FIG. 16 shows experiments using an OVA-induction of murine asthma model. FIG. 16A shows the protocol for asthma induction. FIG. 16B shows pulmonary resistance (R_{L}) as measured in tracheostomized, and ventilated mice. RL is an indicator for airway hyperresponsiveness. FIG. 16C shows BAL cells post antigen sensitization and challenge in comparison with control mice.

FIG. 17 shows the inflammatory response in control and OVA-sensitized asthma model. FIG. 17 shows hematoxylin and eosin (H & E) stain of lungs from PBS- and OVA-sensitized animals. Lungs were stained with H & E stain and imaged under low power (4×). Note peribronchial and perivascular cellular infiltrates in OVA-sensitized animals. Rottlerin-treated, OVA-sensitized/challenged animals demonstrate marked reduction in cellular infiltrates. Images are representative of results from 5-6 animals for each experimental condition.

FIG. 18 shows that a single dose of rottlerin causes reduction in airway resistance in the OVA-asthma model. The experimental conditions of the results shown in FIG. 18A are as follows. Rottlerin (5 μg/g) or PBS was given via the tail vein of mice 5 minutes prior to airway resistance measurements in OVA-challenged/sensitized animals (OVA) and non-sensitized/challenged animals (control). N=8 in each group; *, p<0.05; OVA; rottlerin-treated compared to OVA; PBS-treated. The experimental conditions of the results shown in FIG. 18B are as follows. PBS, Isoproterenol (2.5 μg/g) or Isoproterenol (2.5 μg/g)+Rottlerin (5 μg/g) were given via the tail vein as above.

FIG. 19 shows that rottlerin reduces airway resistance in a house dust mite (HDM) sensitized asthma model. FIG. 19A shows the protocol for asthma induction using the HDM model. The * represents the days when rottlerin was administered I.P during the course of asthma induction. FIG. 19B shows airway hyperresponsiveness (AHR) in mice following HDM challenge and rottlerin administration. (n=4 per group). *p<0.05; HDM/PBS compared to HDM/rottlerin.

FIG. 20 shows that rottlerin inhibits inflammatory response in HDM-exposed mice. H & E stain of lungs from PBS and HDM-exposed animals are shown. Lungs were stained with H & E stain and imaged under low power (4×). Images are representative of similar results from 4 animals for each experimental condition. Note peribronchial and perivascular cellular infiltrates in HDM-exposed animals. Rottlerin-treated, HDM-exposed animals demonstrated marked reduction in cellular infiltrates.

FIG. 21 shows cumulative dose-response curves of WT tracheal rings of OVA-sensitized groups to isoproterenol (ISO) (1 nM to 100 μM) in the presence of PBS, rottlerin or rottlerin+iberiotoxin (IbTX) (n=5 per group). Values are means±S.D. *p<0.05 for PBS compared to rottlerin.

FIG. 22A shows the protocol for an OVA-induced asthma model. Groups of mice received an I.P. injection of OVA/Alum complex on days 0 and 7 and on alternate days 14-22, a 20 minute aerosol challenge of either PBS or 2% (w/v) OVA in PBS, using an ultrasonic nebulizer. FIG. 22B shows that the asthma model exhibited an increase in AHR as shown by an increase in R_{L} in response to MCh.

FIG. 23 shows the ISO-induced increase in outward K⁺ currents in acutely isolated tracheal smooth muscle. FIG. 23A is a diary plot of current recorded during repetitive stimulation by depolarizing ramps every 5 seconds to +200 mV from a holding potential of −20 mV. ISO and ISO+IbTX exposure are indicated by bars at bottom of plot. Dashed line indicates average control current. FIG. 23B shows I-V curves for control, ISO (0.5 mM) and ISO+IbTX (100 nM). Insets demonstrate a series of current traces for voltage steps from a holding potential of −80 mV, with steps from +10 to +220 mV.

FIG. 24 shows the electrophysiological characterization of selected rottlerin derivatives. Two derivatives of rottlerin are shown, methylated rottlerin and reduced rottlerin. FIG. 24A-C show the time course of onset (ON) of the effect of rottlerin or its derivatives (0.5 μM for rottlerin, 1 μM for methylated and reduced rottlerin) and washout (WASH). Electrophysiology was performed using whole-cell patch clamp with a ramp every 5 seconds. FIG. 24D-F show the current traces (insets) from whole cell voltage clamp recordings ([Ca²⁺]ᵢ ˜20 nM for FIG. 24D, 1 μM for FIG. 23E-F) from HEK cells stably expressing BK channels under control conditions and after rottlerin bath application. In FIG. 24F, Δ=wash. G-V curves were generated from tail current analysis for control conditions and after rottlerin or rottlerin-derivative exposure utilizing Boltzmann function.

### DETAILED DESCRIPTION OF THE INVENTION

One embodiment of the present invention is a method of treating or ameliorating the effects of a disease characterized by altered smooth muscle contractility. This method comprises administering to a patient suffering from such a disease an effective amount of a large-conductance Ca²⁺-activated K⁺ (BK) channel modulator.

As used herein, in relation to a disease, the term “characterized by” means one of the characteristics or one of the symptoms of the disease. The term “altered” means different from the norm (i.e. the population at large or an individual not suffering from such a disease). The term “smooth muscle” refers to a group of non-striated muscles, generally found in the walls of the hollow organs of the body (except the heart), including but not limited to the blood vessels, the respiratory tract, the gastrointestinal tract, the bladder, or the uterus. The term “contractility” refers to properties associated with the contraction (e.g., of smooth muscle), such as contraction and relaxation of smooth muscles. The contraction and relaxation of smooth muscles is usually not under voluntary control.

As used herein, a “large-conductance Ca²⁺-activated K⁺ (BK) channel” means an ion channel that conducts potassium (K⁺) ions through cell membranes, and that upon opening or activation, causes transient membrane hyperpolarization, inhibition of Ca²⁺ influx through voltage-dependent Ca²⁺ channels, reduced intracellular concentration of Ca²⁺ and smooth muscle relaxation. A BK channel modulator is a substance that changes the activity or the opening or the closing of the BK channel. Preferably, the BK channel modulator is a BK channel activator. As used herein, “a BK channel activator” means a substance, such as, e.g., all molecules having rottlerin-type activity, that opens the BK channels. BK channel activators may be selected from the group consisting of rottlerin, flindokalner (Bristol-Myers Squibb), BMS-554216 (Bristol-Myers Squibb), Pharmaprojects No. 4420 (Merck & Co) (disclosed in U.S. patent application Ser. No. 09/516,442 filed Dec. 13, 1993), Pharmaprojects No. 4494 (Merck & Co) (disclosed in U.S. patent application Ser. No. 09/519,771 filed Jan. 24, 1994), NS-1619 (NeuroSearch), NSD-551 (NeuroSearch), NS-8 (Nippon Shinyaku), a pharmaceutically acceptable salt thereof, and combinations thereof. Preferably, the BK channel activator is rottlerin.

In the present invention, “rottlerin” is preferably used in an isolated or purified form, either in its keto or enol form. The purified form may be a purified extract from a natural source or a purified compound, which is synthesized. As used herein, “isolated” means that the rottlerin is separated from other components of either (a) a natural source, such as a plant, as disclosed previously herein or (b) a synthetic organic chemical reaction mixture, suitably, via conventional techniques, wherein the rottlerin of the invention is purified. As used herein, “purified” means that when isolated, the isolate contains at least about 20%, including 25%, 30%, 35%, 40%, 45%, 50%, 55%, 60%, 65%, 70%, 75% of rottlerin by weight (wt %) of the isolate. Highly purified rottlerin are also contemplated, wherein the isolate contains at least 80%, preferably at least 90%, such as at least 91, 92, 93, 94, 95, 96, 97, 98, 99 or 100% of rottlerin by weight (wt %) of the isolate. In the present invention, isolate or isolated in regards to rottlerin includes extracts from the native plant, Mallotus phillippinensis (e.g., red kamala powder). Rottlerin may be isolated using methods well-known in the art, such as those published by Anderson and Robertson et al. (Anderson, A. “Kamala resin-rottlerin,” Edin. New Phil. Jour., Vol. 1, pp. 296-300 (1855); Robertson et al., “Rottlerin.” J. Chem. Soc., Part I, pp. 1862-1865 (1937)). Such methods are incorporated by reference as if recited in full herein.

In the present invention, an “effective amount” or “therapeutically effective amount” of a BK channel modulator is an amount of such BK channel modulator that is sufficient to effect beneficial or desired results as described herein when administered to a patient, which is a mammal, preferably a human. A BK channel modulator may also be administered as part of a pharmaceutical composition, such as in a unit dosage form. Preferably, such a unit dosage form is inhaled.

Furthermore, the pharmaceutical composition may be co-administered. In the present invention, “co-administration” includes administration of a pharmaceutical composition comprising a BK channel modulator along with another compound, composition, or pharmaceutical composition together in the same composition, simultaneously in separate compositions, or as separate compositions administered at different times, as deemed most appropriate by a physician.

When a BK channel modulator is co-administered with another compound or composition, that compound or composition is preferably a conventional drug for modulating constriction of ASM such as e.g. corticosteroids, anti-cholinergics, anti-leukotrienes, β-agonists, and/or phosphodiesterase inhibitors. Preferably, the BK channel modulator is co-administered with a β-agonist. Non-limiting examples of a corticosteroid according the present invention include cromolyn sodium, nedocromil, fluticasone, budesonide, triamcinolone, flunisolide, and beclomethasone. A non-limiting example of an anti-cholinergic according the present invention includes ipratropium bromide. Non-limiting examples of an anti-leukotriene according the present invention include montelukast, zafirlukast, and zileuton. Non-limiting examples of a β-agonist according the present invention include albuterol, levalbuterol, salmeterol, formoterol, isoproterenol, and pirbuterol. Non-limiting examples of a phosphodiesterase inhibitor according the present invention include ibudilast, theophylline, CDP840, roflumilast, cilomilast, 4-(3-butoxy-4-methoxyphenyl)methyl-2-imidazolidone (Ro 20-1724), (R)—N-(4-[1-3-cyclopentyloxy-4-methoxyphenyl)-2-(4-pyridyl)ethyl]phenyl)-N′-ethylurea (CT-2450), 6-(4-pyridylmethyl)-8-(3-nitrophenyl)quinoline (PMNPQ), R-rolipram, oglemilast (Glenmark Pharmaceuticals), IPL512602 (Inflazyme pharmaceuticals), N-(3,5-dichloropyrid-4-yl)-[1-(4-fluorobenzyl)-5-hydroxy-indole-3-yl]-glyoxylic acid amide (AWD 12-281), and UK-500001 (Spina, D., PDE4 inhibitors: current status, British J. Pharmacology, 2008. 155:308-315). Co-administration of a BK channel modulator and such drugs leads to synergism (i.e., greater than additive effects). In view of this, lower doses of such drugs may be used in conjunction with a BK channel modulator, which may result in lower overall side effects.

Effective dosage forms, modes of administration, and dosage amounts of, e.g., a BK channel modulator, may be determined empirically, and making such determinations is within the skill of the art. It is understood by those skilled in the art that the dosage amount of, e.g., a BK channel modulator, will vary with the route of administration, the rate of excretion, the duration of the treatment, the identity of any other drugs being administered, the age, size, and species of mammal, e.g., human patient, and like factors well known in the arts of medicine and veterinary medicine. In general, a suitable dose of a rottlerin (or a pharmaceutically acceptable salt thereof) according to the invention will be that amount of the rottlerin (or the pharmaceutically acceptable salt thereof), which is the lowest dose effective to produce the desired effect with no or minimal side effects.

A suitable, non-limiting example of a dosage of a BK channel modulator according to the present invention is from about 10 ng/kg to about 1000 mg/kg, such as from about 1 mg/kg to about 100 mg/kg, including from about 5 mg/kg to about 50 mg/kg, about 1 mg/kg to about 10 mg/kg, about 1 mg/kg to about 3 mg/kg, or about 5 mg/kg to about 7 mg/kg. Other representative dosages of a BK channel modulator include about 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, 100 mg/kg, 125 mg/kg, 150 mg/kg, 175 mg/kg, 200 mg/kg, 250 mg/kg, 300 mg/kg, 400 mg/kg, 500 mg/kg, 600 mg/kg, 700 mg/kg, 800 mg/kg, 900 mg/kg, or 1000 mg/kg. The effective dose of a BK channel modulator maybe administered as two, three, four, five, six or more sub-doses, administered separately at appropriate intervals throughout the day.

In one aspect of this embodiment, diseases characterized by altered smooth muscle contractility include e.g., pneumoconiosis (such as aluminosis, anthracosis, asbestosis, chalicosis, ptilosis, siderosis, silicosis, tabacosis, berylliosis, and byssinosis), chronic obstructive pulmonary disease (COPD), asthma, bronchitis, exacerbation of airway hyperreactivity or cystic fibrosis, cough (including chronic cough), other pulmonary diseases, including other reversible airway diseases, urinary incontinence, and hypertension. Preferably, the disease is asthma, chronic obstructive pulmonary disease, urinary incontinence, or hypertension. More preferably, the disease is asthma.

BK channel modulators disclosed herein may be used to treat acute or chronic diseases according to the methods disclosed herein. As used herein, an “acute” disease means a disease with a rapid onset (i.e., less than 5 minutes) of the symptoms, which may have a dramatic effect on the patient. A non-limiting example of an acute disease is an acute asthma attack, in which the individual may have breathing difficulties and even lose consciousness in an instant. A “chronic” disease means a long-lasting disease or recurrent disease. Chronic asthma is one of many examples of such chronic diseases.

Another embodiment of the present invention is a method of treating or ameliorating the effects of asthma. This method comprises administering to a patient suffering from asthma an effective amount of a BK channel modulator.

A BK channel modulator may also be administered as part of a pharmaceutical composition, such as in a unit dosage form. Preferably, such a unit dosage form is inhaled. Furthermore, the pharmaceutical composition may be co-administered as described above. Preferably, the BK channel modulator is co-administered with a β-agonist.

An additional embodiment of the present invention is a method for decreasing airway constriction and/or airway resistance in a patient without increasing the heart rate of the patient or with no or decreased side effects normally associated with conventional therapy, e.g., tachycardia when β₂ agonists are used. This method comprises administering to the patient an effective of amount of a BK channel modulator or a pharmaceutical composition comprising a pharmaceutically acceptable carrier and a BK channel modulator.

As used herein, “airway constriction” means narrowing of air passages of the lungs, such as from smooth muscle contraction. “Airway resistance” means obstruction to airflow provided by the conducting airways, such as, those found in obstructive lung diseases.

In one aspect of this embodiment, the pharmaceutical composition is in a unit dosage form. Preferably, the unit dosage form is inhaled.

In another aspect of this embodiment, the pharmaceutical composition may be co-administered as described above. Preferably, the BK channel modulator is co-administered with a β-agonist. It is noted that by using the methods of the present invention, lower levels of the co-administered composition, e.g., β-agonists, may be used; thus reducing the possible side effects associated with the use of such composition.

A further embodiment of the present invention is a method for modulating inflammation in a lung of a patient. This method comprises administering to a patient an effective of amount of a BK channel modulator or a pharmaceutical composition comprising a BK channel modulator, which amount is sufficient to modulate the inflammation.

As used herein in relation to inflammation, “modulating”, “modulation” and like terms mean to increase or, preferably, to decrease inflammation of the lung of a patient administered a compound or pharmaceutical composition according to the present invention relative to a patient who is not administered the compound or the pharmaceutical composition.

In one aspect of this embodiment, a BK channel modulator or a pharmaceutical composition comprising a BK channel modulator is administered in a unit dosage form by e.g., inhalation. In another aspect of this embodiment, a BK channel modulator or a pharmaceutical composition comprising a BK channel modulator may be co-administered as described above. Preferably, the BK channel modulator is co-administered with a β-agonist.

Yet another embodiment of the present invention is pharmaceutical composition for treating or ameliorating the effects of a disease characterized by altered smooth muscle contractility. This pharmaceutical composition comprises a pharmaceutically acceptable carrier and a BK channel modulator.

In one aspect of this embodiment, diseases characterized by altered smooth muscle contractility include e.g., pneumoconiosis (such as aluminosis, anthracosis, asbestosis, chalicosis, ptilosis, siderosis, silicosis, tabacosis, berylliosis, and byssinosis), chronic obstructive pulmonary disease (COPD), asthma, bronchitis, exacerbation of airway hyperreactivity or cystic fibrosis, cough (including chronic cough), other pulmonary diseases, including other reversible airway diseases, urinary incontinence, and hypertension. Preferably, the disease is asthma, chronic obstructive pulmonary disease, urinary incontinence, or hypertension. More preferably, the disease is asthma.

In another aspect of this embodiment, the pharmaceutical composition is in a unit dosage form. Preferably, the unit dosage form is inhaled.

In yet another aspect of this embodiment, the pharmaceutical composition is co-administered as described above. Preferably, the BK channel modulator is co-administered with a β-agonist.

An additional embodiment of the present invention is a pharmaceutical composition for treating or ameliorating the effects of asthma. This pharmaceutical composition comprises a pharmaceutically acceptable carrier and a BK channel modulator.

A compound or pharmaceutical composition of the present invention may be administered in any desired and effective manner. Preferably, the compound or pharmaceutical composition of the present invention is administered to a patient in need thereof through a mucosal lining, by, e.g., a nasal or pulmonary spray.

Thus, compounds and pharmaceutical compositions according to the present invention may be administered in an aqueous solution as a nasal or pulmonary spray and may be dispensed in spray form by a variety of methods known to those skilled in the art. Exemplary systems for dispensing liquids as a nasal spray are disclosed in U.S. Pat. No. 4,511,069. The formulations may be presented in multi-dose containers, for example in the sealed dispensing system disclosed in U.S. Pat. No. 4,511,069. Additional aerosol delivery forms may include, e.g., compressed air-, jet-, ultrasonic-, and piezoelectric nebulizers, which deliver the compound or pharmaceutical composition according to the present invention dissolved or suspended in a pharmaceutical solvent, e.g., water, ethanol, or a mixture thereof.

For example, a nebulizer may be selected on the basis of allowing the formation of an aerosol of a BK channel modulator disclosed herein. The delivered amount of a BK channel modulator provides a therapeutic effect for the diseases disclosed herein. The nebulizer may deliver an aerosol comprising a mass median aerodynamic diameter from about 2 microns to about 5 microns with a geometric standard deviation less than or equal to about 2.5 microns, a mass median aerodynamic diameter from about 2.5 microns to about 4.5 microns with a geometric standard deviation less than or equal to about 1.8 microns, and a mass median aerodynamic diameter from about 2.8 microns to about 4.3 microns with a geometric standard deviation less than or equal to about 2 microns. In other instances, the aerosol can be produced using a vibrating mesh nebulizer. An example of a vibrating mesh nebulizer includes the PARI E-FLOW™ nebulizer or a nebulizer using PARI eFlow technology. More examples of nebulizers are provided in U.S. Pat. Nos. 4,268,460; 4,253,468; 4,046,146; 3,826,255; 4,649,911; 4,510,929; 4,624,251; 5,164,740; 5,586,550; 5,758,637; 6,644,304; 6,338,443; 5,906,202; 5,934,272; 5,960,792; 5,971,951; 6,070,575; 6,192,876; 6,230,706; 6,349,719; 6,367,470; 6,543,442; 6,584,971; 6,601,581; 4,263,907; 5,709,202; 5,823,179; 6,192,876; 6,644,304; 5,549,102; 6,083,922; 6,161,536; 6,264,922; 6,557,549; and 6,612,303; all of which are hereby incorporated by reference in their entireties. More commercial examples of nebulizers that can be used with the BK channel modulators described herein include Respirgard II™, Aeroneb™, Aeroneb™ Pro, and Aeroneb™ Go produced by Aerogen; AERx™ and AERx Essence™ produced by Aradigm; Porta-Neb™, Freeway Freedom™, Sidestream, Ventstream and I-neb produced by Respironics, Inc. (Murrysville, Pa.); and PARI LC-Plus™, PARI LC-Start, produced by PARI Respiratory Equipment Inc. (Midlothian, Va.). By further non-limiting example, U.S. Pat. No. 6,196,219, is hereby incorporated by reference in its entirety.

A suitable, non-limiting example of a dosage of a BK channel modulator according to the present invention administered via a nebulizer to an adult human may be from about 0.1 mg/m²/day to 100 mg/m²/day, such as from about 0.5 mg/m²/day to about 80 mg/m²/day, including from about 1 mg/m²/day to about 50 mg/m²/day, about 1 mg/m²/day to about 20 mg/m²/day, about 1 mg/m²/day to about 10 mg/m²/day, about 1 mg/m²/day to about 7 mg/m²/day, or about 3 mg/m²/day to about 7 mg/m²/day. Other representative dosages of a BK channel modulator include about 0.1 mg/m²/day, 0.2 mg/m²/day, 0.3 mg/m²/day, 0.4 mg/m²/day 0.5 mg/m²/day, 0.6 mg/m²/day, 0.7 mg/m²/day, 0.8 mg/m²/day, 0.9 mg/m²/day, 1 mg/m²/day, 2 mg/m²/day, 3 mg/m²/day, 4 mg/m²/day, 5 mg/m²/day, 6 mg/m²/day, 7 mg/m²/day, 8 mg/m²/day, 9 mg/m²/day, 10 mg/m²/day, 11 mg/m²/day, 12 mg/m²/day, 13 mg/m²/day, 14 mg/m²/day, 15 mg/m²/day, 16 mg/m²/day, 17 mg/m²/day, 18 mg/m²/day, 19 mg/m²/day, 20 mg/m²/day, 25 mg/m²/day, 30 mg/m²/day, 35 mg/m²/day, 40 mg/m²/day, 45 mg/m²/day, 50 mg/m²/day, 55 mg/m²/day, 60 mg/m²/day, 65 mg/m²/day, 70 mg/m²/day, 75 mg/m²/day 80 mg/m²/day, 85 mg/m²/day, 90 mg/m²/day, 95 mg/m²/day, or 100 mg/m²/day. Dosages may be reduced in a child. The effective dose of a BK channel modulator maybe administered as two, three, four, five, six or more sub-doses, administered separately at appropriate intervals throughout the day.

Nasal and pulmonary spray solutions of the present invention typically comprise the compound or pharmaceutical composition to be delivered, optionally formulated with a surface-active agent, such as a nonionic surfactant (e.g., polysorbate-80), and one or more buffers. In some embodiments of the present invention, the nasal spray solution further comprises a propellant. The pH of the nasal spray solution is optionally between about pH 3.0 and 6.0, preferably 5.0.+/−0.3. Suitable buffers for use within these compositions are as described herein or as otherwise known in the art. Other components may be added to enhance or maintain chemical stability, including preservatives, surfactants, dispersants, or gases. Suitable preservatives include, but are not limited to, phenol, methyl paraben, paraben, m-cresol, thiomersal, chlorobutanol, benzylalkonimum chloride, and the like. Suitable surfactants include, but are not limited to, oleic acid, sorbitan trioleate, polysorbates, lecithin, phosphotidyl cholines, and various long chain diglycerides and phospholipids. Suitable dispersants include, but are not limited to, ethylenediaminetetraacetic acid, and the like. Suitable gases include, but are not limited to, nitrogen, helium, chlorofluorocarbons (CFCs), hydrofluorocarbons (HFCs), carbon dioxide, air, and the like.

Within alternate embodiments, mucosal formulations of the present invention may be administered as dry powder formulations comprising the compound or pharmaceutical composition according to the present invention in a dry, usually lyophilized, form of an appropriate particle size, or within an appropriate particle size range, for intranasal delivery. Minimum particle size appropriate for deposition within the nasal or pulmonary passages is often about 0.5 μm mass median equivalent aerodynamic diameter (MMEAD), commonly about 1 μm MMEAD, and more typically about 2 μm MMEAD. Maximum particle size appropriate for deposition within the nasal passages is often about 10 μm MMEAD, commonly about 8 μm MMEAD, and more typically about 4 μm MMEAD. Intranasally respirable powders within these size ranges can be produced by a variety of conventional techniques, such as jet milling, spray drying, solvent precipitation, supercritical fluid condensation, and the like. These dry powders of appropriate MMEAD can be administered to a patient via a conventional dry powder inhaler (DPI), which rely on the patient's breath, upon pulmonary or nasal inhalation, to disperse the power into an aerosolized amount. Alternatively, the dry powder may be administered via air-assisted devices that use an external power source to disperse the powder into an aerosolized amount, e.g., a piston pump.

Dry powder devices typically require a powder mass in the range from about 1 mg to 20 mg to produce a single aerosolized dose (“puff”). If the required or desired dose of the compound or pharmaceutical composition according to the present invention is lower than this amount, the powdered active agent will typically be combined with a pharmaceutical dry bulking powder to provide the required total powder mass. Preferred dry bulking powders include sucrose, lactose, dextrose, mannitol, glycine, trehalose, human serum albumin (HSA), and starch. Other suitable dry bulking powders include cellobiose, dextrans, maltotriose, pectin, sodium citrate, sodium ascorbate, and the like.

To formulate compositions for mucosal delivery within the present invention, the compound or pharmaceutical composition according to the present invention can be combined with various pharmaceutically acceptable additives, as well as a base or carrier for dispersion of the active agent(s). Desired additives include, but are not limited to, pH control agents, such as arginine, sodium hydroxide, glycine, hydrochloric acid, citric acid, etc. In addition, local anesthetics (e.g., benzyl alcohol), isotonizing agents (e.g., sodium chloride, mannitol, sorbitol), adsorption inhibitors (e.g., Tween 80), solubility enhancing agents (e.g., cyclodextrins and derivatives thereof), stabilizers (e.g., serum albumin), and reducing agents (e.g., glutathione) can be included. When the composition for mucosal delivery is a liquid, the tonicity of the formulation, as measured with reference to the tonicity of 0.9% (w/v) physiological saline solution taken as unity, is typically adjusted to a value at which no substantial, irreversible tissue damage will be induced in the nasal mucosa at the site of administration. Generally, the tonicity of the solution is adjusted to a value of about ⅓ to 3, more typically ½ to 2, and most often ¾ to 1.7.

The compounds or compositions of the present invention may be dispersed in a base or vehicle, which may comprise a hydrophilic compound having a capacity to disperse the compounds or compositions of the present invention and any desired additives. The base may be selected from a wide range of suitable carriers, including but not limited to, copolymers of polycarboxylic acids or salts thereof, carboxylic anhydrides (e.g. maleic anhydride) with other monomers (e.g. methyl (meth)acrylate, acrylic acid, etc.), hydrophilic vinyl polymers such as polyvinyl acetate, polyvinyl alcohol, polyvinylpyrrolidone, cellulose derivatives such as hydroxymethylcellulose, hydroxypropylcellulose, etc., and natural polymers such as chitosan, collagen, sodium alginate, gelatin, hyaluronic acid, and nontoxic metal salts thereof. Often, a biodegradable polymer is selected as a base or carrier, for example, polylactic acid, poly(lactic acid-glycolic acid) copolymer, polyhydroxybutyric acid, poly(hydroxybutyric acid-glycolic acid) copolymer and mixtures thereof. Alternatively or additionally, synthetic fatty acid esters such as polyglycerin fatty acid esters, sucrose fatty acid esters, etc. can be employed as carriers. Hydrophilic polymers and other carriers can be used alone or in combination, and enhanced structural integrity can be imparted to the carrier by partial crystallization, ionic bonding, crosslinking and the like. The carrier can be provided in a variety of forms, including, fluid or viscous solutions, gels, pastes, powders, microspheres and films for direct application to the nasal mucosa. The use of a selected carrier in this context may result in promotion of absorption of the compound or composition according to the present invention.

The compounds or compositions of the present invention can be combined with the base or carrier according to a variety of methods, and release of the compounds or compositions of the present invention may be by diffusion, disintegration of the carrier, or associated formulation of water channels. In some circumstances, the active agent is dispersed in microcapsules (microspheres) or nanocapsules (nanospheres) prepared from a suitable polymer, e.g., isobutyl 2-cyanoacrylate and dispersed in a biocompatible dispersing medium applied to the nasal mucosa, which yields sustained delivery and biological activity over a protracted time.

To further enhance mucosal delivery of compounds or compositions of the present invention, formulations comprising such agents may also contain a hydrophilic low molecular weight compound as a base or excipient. Such hydrophilic low molecular weight compounds provide a passage medium through which a water-soluble active agent, such as a physiologically active peptide or protein, may diffuse through the base to the body surface where the active agent is absorbed. The hydrophilic low molecular weight compound optionally absorbs moisture from the mucosa or the administration atmosphere and dissolves the water-soluble active peptide. The molecular weight of the hydrophilic low molecular weight compound is generally not more than 10,000 and preferably not more than 3,000. Exemplary hydrophilic low molecular weight compounds include polyol compounds, such as oligo-, di- and monosaccarides such as sucrose, mannitol, sorbitol, lactose, L-arabinose, D-erythrose, D-ribose, D-xylose, D-mannose, trehalose, D-galactose, lactulose, cellobiose, gentibiose, glycerin and polyethylene glycol. Other examples of hydrophilic low molecular weight compounds useful as carriers within the invention include N-methylpyrrolidone, and alcohols (e.g. oligovinyl alcohol, ethanol, ethylene glycol, propylene glycol, etc.) These hydrophilic low molecular weight compounds can be used alone or in combination with one another or with other active or inactive components of the intranasal formulation.

In sum, mucosal administration according to the invention allows effective self-administration of treatment by patients, provided that sufficient safeguards are in place to control and monitor dosing and side effects. Mucosal administration also overcomes certain drawbacks of other administration forms, such as injections, that are painful and expose the patient to possible infections and may present drug bioavailability problems. For nasal and pulmonary delivery, systems for controlled aerosol dispensing of therapeutic liquids as a spray are well known. For example, metered doses of a compound or composition of the present invention are delivered by means of a specially constructed mechanical pump valve, U.S. Pat. No. 4,511,069.

In the present invention, other methods of delivery may also be used. Such methods include, for example, administration by oral ingestion, or as an ointment or drop for local administration to the eyes, or for parenteral or other administration in any appropriate manner such as intraperitoneal, subcutaneous, topical, intradermal, rectal, vaginal, sublingual, intramuscular, intravenous, intraarterial, intrathecal, or intralymphatic. Further, a pharmaceutical composition of the present invention may be administered in conjunction with other treatments. A pharmaceutical composition of the present invention may be encapsulated or otherwise protected against gastric or other secretions, if desired.

The pharmaceutically acceptable compositions of the invention comprise one or more active ingredients in admixture with one or more pharmaceutically-acceptable carriers and, optionally, one or more other compounds, drugs, ingredients and/or materials. Regardless of the route of administration selected, the agents/compounds of the present invention are formulated into pharmaceutically-acceptable dosage forms by conventional methods known to those of skill in the art. See, e.g., Remington, The Science and Practice of Pharmacy (21^{st} Edition, Lippincott Williams and Wilkins, Philadelphia, Pa.).

Pharmaceutically acceptable carriers are well known in the art (see, e.g., Remington, The Science and Practice of Pharmacy (21^{st} Edition, Lippincott Williams and Wilkins, Philadelphia, Pa.) and The National Formulary (American Pharmaceutical Association, Washington, D.C.)) and include sugars (e.g., lactose, sucrose, mannitol, and sorbitol), starches, cellulose preparations, calcium phosphates (e.g., dicalcium phosphate, tricalcium phosphate and calcium hydrogen phosphate), sodium citrate, water, aqueous solutions (e.g., saline, sodium chloride injection, Ringer's injection, dextrose injection, dextrose and sodium chloride injection, lactated Ringer's injection), alcohols (e.g., ethyl alcohol, propyl alcohol, and benzyl alcohol), polyols (e.g., glycerol, propylene glycol, and polyethylene glycol), organic esters (e.g., ethyl oleate and tryglycerides), biodegradable polymers (e.g., polylactide-polyglycolide, poly(orthoesters), and poly(anhydrides)), elastomeric matrices, liposomes, microspheres, oils (e.g., corn, germ, olive, castor, sesame, cottonseed, and groundnut), cocoa butter, waxes (e.g., suppository waxes), paraffins, silicones, talc, silicylate, etc. Each pharmaceutically acceptable carrier used in a pharmaceutical composition of the invention must be “acceptable” in the sense of being compatible with the other ingredients of the formulation and not injurious to the subject. Carriers suitable for a selected dosage form and intended route of administration are well known in the art, and acceptable carriers for a chosen dosage form and method of administration can be determined using ordinary skill in the art.

The pharmaceutical compositions of the invention may, optionally, contain additional ingredients and/or materials commonly used in such pharmaceutical compositions. These ingredients and materials are well known in the art and include (1) fillers or extenders, such as starches, lactose, sucrose, glucose, mannitol, and silicic acid; (2) binders, such as carboxymethylcellulose, alginates, gelatin, polyvinyl pyrrolidone, hydroxypropylmethyl cellulose, sucrose and acacia; (3) humectants, such as glycerol; (4) disintegrating agents, such as agar-agar, calcium carbonate, potato or tapioca starch, alginic acid, certain silicates, sodium starch glycolate, cross-linked sodium carboxymethyl cellulose and sodium carbonate; (5) solution retarding agents, such as paraffin; (6) absorption accelerators, such as quaternary ammonium compounds; (7) wetting agents, such as cetyl alcohol and glycerol monostearate; (8) absorbents, such as kaolin and bentonite clay; (9) lubricants, such as talc, calcium stearate, magnesium stearate, solid polyethylene glycols, and sodium lauryl sulfate; (10) suspending agents, such as ethoxylated isostearyl alcohols, polyoxyethylene sorbitol and sorbitan esters, microcrystalline cellulose, aluminum metahydroxide, bentonite, agar-agar and tragacanth; (11) buffering agents; (12) excipients, such as lactose, milk sugars, polyethylene glycols, animal and vegetable fats, oils, waxes, paraffins, cocoa butter, starches, tragacanth, cellulose derivatives, polyethylene glycol, silicones, bentonites, silicic acid, talc, salicylate, zinc oxide, aluminum hydroxide, calcium silicates, and polyamide powder; (13) inert diluents, such as water or other solvents; (14) preservatives; (15) surface-active agents; (16) dispersing agents; (17) control-release or absorption-delaying agents, such as hydroxypropylmethyl cellulose, other polymer matrices, biodegradable polymers, liposomes, microspheres, aluminum monosterate, gelatin, and waxes; (18) opacifying agents; (19) adjuvants; (20) wetting agents; (21) emulsifying and suspending agents; (22), solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan; (23) propellants as disclosed above, such as hydrofluoroalkane, particularly 1,1,1,2-tetrafluoroethane, heptafluoralkane (HFA) such as 1,1,1,2,3,3,3-heptafluoro-n-propane or mixtures thereof, as well as other chlorofluorohydrocarbons and other volatile unsubstituted hydrocarbons, such as butane and propane; (24) antioxidants; (25) agents which render the formulation isotonic with the blood of the intended recipient, such as sugars and sodium chloride; (26) thickening agents; (27) coating materials, such as lecithin; and (28) sweetening, flavoring, coloring, perfuming and preservative agents. Each such ingredient or material must be “acceptable” in the sense of being compatible with the other ingredients of the formulation and not injurious to the subject. Ingredients and materials suitable for a selected dosage form and intended route of administration are well known in the art, and acceptable ingredients and materials for a chosen dosage form and method of administration may be determined using ordinary skill in the art.

Pharmaceutical compositions suitable for oral administration may be in the form of capsules, cachets, pills, tablets, powders, granules, a solution or a suspension in an aqueous or non-aqueous liquid, an oil-in-water or water-in-oil liquid emulsion, an elixir or syrup, a pastille, a bolus, an electuary or a paste. These formulations may be prepared by methods known in the art, e.g., by means of conventional pan-coating, mixing, granulation or lyophilization processes.

Solid dosage forms for oral administration (capsules, tablets, pills, dragees, powders, granules and the like) may be prepared, e.g., by mixing the active ingredient(s) with one or more pharmaceutically-acceptable carriers and, optionally, one or more fillers, extenders, binders, humectants, disintegrating agents, solution retarding agents, absorption accelerators, wetting agents, absorbents, lubricants, and/or coloring agents. Solid compositions of a similar type may be employed as fillers in soft and hard-filled gelatin capsules using a suitable excipient. A tablet may be made by compression or molding, optionally with one or more accessory ingredients. Compressed tablets may be prepared using a suitable binder, lubricant, inert diluent, preservative, disintegrant, surface-active or dispersing agent. Molded tablets may be made by molding in a suitable machine. The tablets, and other solid dosage forms, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein. They may be sterilized by, for example, filtration through a bacteria-retaining filter. These compositions may also optionally contain opacifying agents and may be of a composition such that they release the active ingredient only, or preferentially, in a certain portion of the gastrointestinal tract, optionally, in a delayed manner. The active ingredient can also be in microencapsulated form.

Liquid dosage forms for oral administration include pharmaceutically-acceptable emulsions, microemulsions, solutions, suspensions, syrups and elixirs. The liquid dosage forms may contain suitable inert diluents commonly used in the art. Besides inert diluents, the oral compositions may also include adjuvants, such as wetting agents, emulsifying and suspending agents, sweetening, flavoring, coloring, perfuming and preservative agents. Suspensions may contain suspending agents.

Pharmaceutical compositions for rectal or vaginal administration may be presented as a suppository, which may be prepared by mixing one or more active ingredient(s) with one or more suitable nonirritating carriers which are solid at room temperature, but liquid at body temperature and, therefore, will melt in the rectum or vaginal cavity and release the active compound. Pharmaceutical compositions which are suitable for vaginal administration also include pessaries, tampons, creams, gels, pastes, foams or spray formulations containing such pharmaceutically-acceptable carriers as are known in the art to be appropriate.

Dosage forms for the topical or transdermal administration include powders, sprays, ointments, pastes, creams, lotions, gels, solutions, patches, drops and inhalants as previously disclosed. The active agent(s)/compound(s) may be mixed under sterile conditions with a suitable pharmaceutically-acceptable carrier. The ointments, pastes, creams and gels may contain excipients. Powders and sprays may contain excipients and propellants as previously disclosed.

Pharmaceutical compositions suitable for parenteral administrations comprise one or more agent(s)/compound(s) in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain suitable antioxidants, buffers, solutes which render the formulation isotonic with the blood of the intended recipient, or suspending or thickening agents. Proper fluidity can be maintained, for example, by the use of coating materials, by the maintenance of the required particle size in the case of dispersions, and by the use of surfactants. These compositions may also contain suitable adjuvants, such as wetting agents, emulsifying agents and dispersing agents. It may also be desirable to include isotonic agents. In addition, prolonged absorption of the injectable pharmaceutical form may be brought about by the inclusion of agents which delay absorption.

In some cases, in order to prolong the effect of a drug (e.g., pharmaceutical formulation), it is desirable to slow its absorption from subcutaneous or intramuscular injection. This may be accomplished by the use of a liquid suspension of crystalline or amorphous material having poor water solubility.

The rate of absorption of the active agent/drug then depends upon its rate of dissolution which, in turn, may depend upon crystal size and crystalline form. Alternatively, delayed absorption of a parenterally-administered agent/drug may be accomplished by dissolving or suspending the active agent/drug in an oil vehicle. Injectable depot forms may be made by forming microencapsule matrices of the active ingredient in biodegradable polymers. Depending on the ratio of the active ingredient to polymer, and the nature of the particular polymer employed, the rate of active ingredient release can be controlled. Depot injectable formulations are also prepared by entrapping the drug in liposomes or microemulsions which are compatible with body tissue. The injectable materials can be sterilized for example, by filtration through a bacterial-retaining filter.

The formulations may be presented in unit-dose or multi-dose sealed containers, for example, ampules and vials, and may be stored in a lyophilized condition requiring only the addition of the sterile liquid carrier, for example water for injection, immediately prior to use. Extemporaneous injection solutions and suspensions may be prepared from sterile powders, granules and tablets of the type described above.

As described above, BK_{Ca} channels play a critical role in modulating neuronal processes and smooth muscle contractile tone. Accordingly, BK_{Ca} regulation has significant implications in the study of diseases in which smooth muscle contraction may be abnormal. Alteration of the channel's activity by phosphorylation represents an important regulatory pathway leading to modulation of cellular excitability. The present inventors have herein demonstrated that pharmacologic approaches to activate BK_{Ca} channels represent an emerging novel strategy to control membrane excitability.

Thus, in another aspect, the present invention relates to several findings, concerning BK_{Ca} regulation. In particular, the inventors have discovered that rottlerin dramatically increases BK_{Ca} channel activity in a non-Ca²⁺ dependent, but reversible fashion. Moreover, rottlerin's mechanism appears unique: tail currents are markedly prolonged after exposure to rottlerin, implying a slowing of deactivation and the G-V curve is reversibly shifted by more than 100 mV to the left. Similar results were observed in a rat BK_{Ca} channel heterologously expressed in HEK293. Accordingly, the present invention provides compositions and methods for regulating the BK channel using rottlerin and derivatives thereof.

More specifically, the present invention also encompasses compositions and methods for treating or preventing BK channel mediated disorders by administering to a subject an effective amount of a BK channel activator, including but not limited to rottlerin and derivatives thereof.

As used herein, a BK channel or BK_{Ca} channel mediated disorder refers to disorders related to under or over activation of the BK channel. For purposes of the present invention, such disorders include, but are not limited to, hypertension, asthma, urinary incontinence, gastroenteric hypermotility, coronary spasm, pulmonary disease, psychoses, convulsion, anxiety, erectile dysfunction and neurologic dysfunction.

The term “derivative” as used herein refers to a chemical compound that is structurally similar to another and may be theoretically derivable from it, but differs slightly in composition, but has the same or better activity and safety profile. For example, an analogue of rottlerin is a compound that differs slightly from rottlerin (e.g., as in the replacement of one atom by an atom of a different element or in the presence of a particular functional group), and may be derivable from rottlerin.

In one aspect of the present invention, a compound for use in modulating BK channel activity is provided having the general formula:

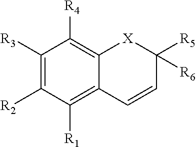

wherein X is CH₂, O, N, or S; R₁ and R₃ are independently selected from H, OH, NH or SH; R₂ is ethanone, acetyl, alkenyl, aryl or alkyl; R₄ is CO-[(E)CHCH]ₙ-Ph, CN-[(E)CHCH]ₙ-Ph, or COOZ, wherein Z is alkenyl, aryl, or alkyl; and R₅ and R₆ are independently selected from H, OH, NH, SH, alkenyl, aryl, or alkyl. In one embodiment, the compound is rottlerin or a derivative thereof.

The present invention also provides a pharmaceutical composition comprising the above-described compound, or a derivative thereof, and optionally, a pharmaceutically acceptable carrier, for use in treating or preventing a BK channel associated disorder. In a specific embodiment, the compound is rottlerin.

The term “treating,” as used herein in relation to a disorder (as opposed to the effects of a disorder), includes treating any one or more of the conditions underlying or characteristic of a particular disorder. As used herein, the term “preventing” in relation to a disorder (as opposed to the effects of a disorder), includes preventing the initiation of a particular disorder, delaying the initiation the disorder, preventing the progression or advancement of the disorder, slowing the progression or advancement of the disorder, delaying the progression or advancement of the disorder, and reversing the progression of the disorder from an advanced to a less advanced stage.

By way of example, in an embodiment of the invention, hypertension is treated in a subject in need of treatment by administering to the subject a therapeutically effective amount of rottlerin or a derivative thereof, which amount is effective to treat the hypertension. The subject is preferably a mammal (e.g., humans, domestic animals, and commercial animals, including cows, dogs, monkeys, mice, pigs, and rats), and is most preferably a human.

In another aspect of the present invention, the above-described compounds and pharmaceutical compositions can be used to regulate membrane excitability both in vitro and in vivo. In one example, the compounds and pharmaceutical compositions of the present invention can be used to treat or prevent a hyperexcitability disorder. In an embodiment of the invention, the hyperexcitability disorder is asthma. In another embodiment of the invention, the hyperexcitability disorder is hypertension. In other embodiments of the present invention, the hyperexcitability disorder includes, but is not necessarily limited to, urinary incontinence, gastroenteric hypermotility, coronary spasm, psychoses, convulsion and anxiety. In another embodiment, the compounds and pharmaceutical compositions of the present invention are used in treating or preventing erectile dysfunction. In yet other embodiments, the compounds and pharmaceutical compositions of the present invention are used in treating or preventing coronary artery vasospasm. In another embodiment, the compounds and pharmaceutical compositions of the present invention are used in treating or preventing neurologic dysfunction. In another embodiment, the compounds and pharmaceutical compositions of the present invention are used in post-stroke neuroprotection.

The present invention also provides methods for treating or preventing a hyperexcitability disorder in a subject, comprising administering to the subject a therapeutically effective amount of the pharmaceutical composition of the invention. In an embodiment of the invention, the hyperexcitability disorder includes, but is not necessarily limited to, asthma, urinary incontinence, gastroenteric hypermotility, hypertension, coronary spasm, psychoses, convulsion and anxiety.

The present invention also provides methods for treating or preventing erectile dysfunction in a subject by administering to the subject a therapeutically effective amount of the pharmaceutical compositions of the invention. Additionally, the present invention also provides methods for treating or preventing a coronary artery vasospasm in a subject, comprising administering to the subject a therapeutically effective amount of a pharmaceutical composition of the present invention.

The present invention additionally provides methods for treating or preventing hypertension in a subject, comprising administering to the subject a therapeutically effective amount of a pharmaceutical composition of the present invention. As used in the context of the present invention, hypertension refers to a condition characterized by an increased systolic and/or diastolic blood pressure. By way of non-limiting example, hypertension in a human subject is characterized by a systolic pressure above 140 mm Hg and/or a diastolic pressure above 90 mm Hg.

The present invention also provides methods for treating or preventing a neurologic dysfunction in a subject, comprising administering to the subject a therapeutically effective amount of a pharmaceutical composition of the present invention. The present invention also provides methods for post-stroke neuroprotection in a subject by administering a therapeutically effective amount of a pharmaceutical composition of the present invention.

The present invention further provides kits for use in treating or preventing hyperexcitability disorders comprising an effective amount of a pharmaceutical composition of the present invention, optionally, in association with a pharmaceutically acceptable carrier. In an embodiment, the hyperexcitability disorder includes, but is not necessarily limited to, asthma, urinary incontinence, gastroenteric hypermotility, hypertension, coronary spasm, psychoses, convulsion and anxiety.

The present invention also provides kits for use in treating or preventing erectile dysfunction, coronary artery vasospasm, hypertension or neurologic dysfunction in a subject, comprising administering a therapeutically effective amount of a pharmaceutical composition of the present invention.

Finally, the present invention also provides kits for use in post-stroke neuroprotection in a subject, comprising a therapeutically effective amount of a pharmaceutical composition of the present invention.

As noted above, rottlerin (5,7-dihydroxy-2,2-dimethyl-6-(2,4,6-trihydroxy-3-methyl-5-acetylbenzyl)-8-cinnamoyl-I,2-chromene), and derivatives thereof, have been frequently, but incorrectly, characterized in the literature as PKCδ inhibitors. The present invention establishes for the first time that rottlerin and its derivatives can be used to activate the BK_{Ca} channel. This new therapy will provide unique strategies to treat and prevent a variety of disorders mediated by BK_{Ca} channel activity.

Methods of preparing rottlerin and its derivatives are well known in the art. Rottlerin, for example, is commercially available from A.G. Scientific, Inc., 6450 Lusk Blvd: Suite E 102, San Diego, Calif. 92121. Rottlerin and derivatives thereof may be synthesized in accordance with known organic chemistry procedures that are readily understood by those of skill in the art. The term “synthesize” as used in the present invention refers to formation of a particular chemical compound from its constituent parts using synthesis processes known in the art. Such synthesis processes include, for example, the use of light, heat, chemical, enzymatic or other means to form particular chemical composition.

In a method of the present invention, a composition comprising rottlerin or a derivative thereof may be administered to a subject in combination with another BK_{Ca} channel activator, such that a synergistic therapeutic effect is produced. A “synergistic therapeutic effect” refers to a greater-than-additive therapeutic effect which is produced by a combination of two therapeutic agents, and which exceeds that which would otherwise result from individual administration of either therapeutic agent alone. For instance, administration of rottlerin in combination with a derivative thereof unexpectedly results in a synergistic therapeutic effect by providing greater efficacy than would result from use of either of therapeutic agents alone. Rottlerin enhances the effect of the rottlerin derivative. Therefore, lower doses of one or both of the therapeutic agents may be used in treating for example, hypertension, resulting in increased therapeutic efficacy and decreased side-effects.

The invention also provides compositions and methods for treating or preventing neuronal damage in a post-stroke subject comprising administering to the subject a therapeutically effective amount of rottlerin or derivatives thereof.

The present invention further provides kits for use in treating or preventing hyperexcitability disorders in a subject comprising a therapeutically effective amount of a pharmaceutical composition of the present invention, optionally, in combination with a pharmaceutically acceptable carrier. In an embodiment, the hyperexcitability disorder includes, but is not necessarily limited to, asthma, urinary incontinence, gastroenteric hypermotility, hypertension, coronary spasm, psychoses, convulsion and anxiety.

The present invention also provides kits for use in treating or preventing erectile dysfunction, coronary artery vasospasm, hypertension or neurologic dysfunction in a subject, comprising administering a therapeutically effective amount of a pharmaceutical composition of the present invention. The present invention further provides kits for use in treating or preventing hyperexcitability disorders in a subject comprising a therapeutically effective amount of a pharmaceutical composition of the present invention, optionally, in combination with a pharmaceutically acceptable carrier. In an embodiment, the hyperexcitability disorder includes, but is not necessarily limited to, asthma, urinary incontinence, gastroenteric hypermotility, hypertension, coronary spasm, psychoses, convulsion, and anxiety.

The present invention also provides pharmaceutical compositions for use in treating or preventing erectile dysfunction, coronary artery vasospasm, hypertension or neurologic dysfunction in a subject, comprising administering a therapeutically effective amount of a pharmaceutical composition of the present invention.

Finally, the present invention also provides kits for use in post-stroke neuroprotection in a subject, comprising a therapeutically effective amount of a pharmaceutical composition of the present invention.

The following examples are provided to further illustrate the compositions and methods of the present invention. These examples are illustrative only and are not intended to limit the scope of the invention in any way.

### EXAMPLES

### Example 1

### Rottlerin Activates BK_{Ca} Channel

BK_{Ca} regulation has significant implications in the study of diseases in which smooth muscle contraction may be abnormal. BK_{Ca} can be potently regulated by PKC activating vasoconstrictors. In order to elucidate the functional effects of PKC phosphorylation, the inventors evaluated putative PKC inhibitory compounds under non-phosphorylated conditions. Thus, it is expected that these PKC inhibitors should have no effect in this study.

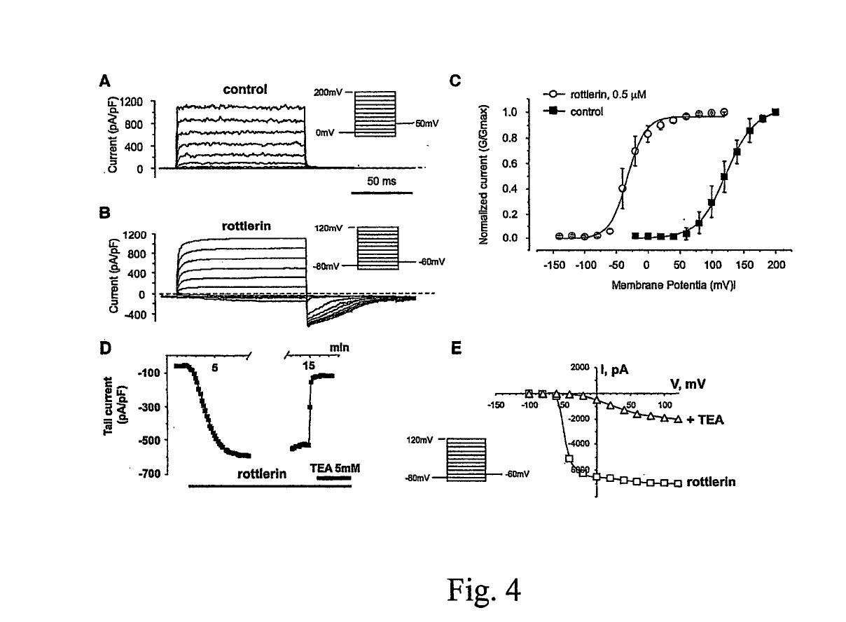

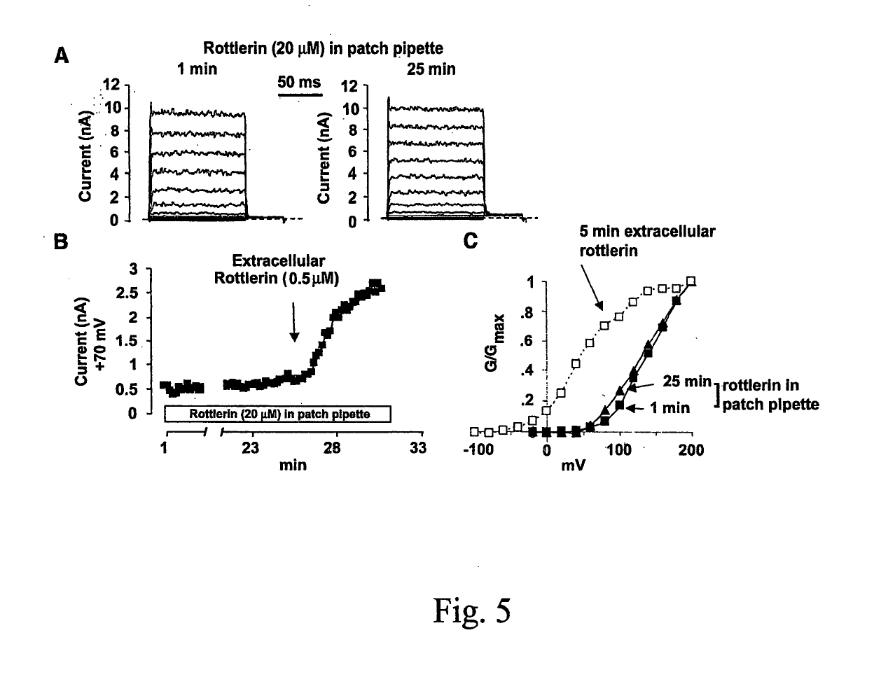

Surprisingly, one compound, rottlerin (<100 nM) dramatically increased channel activity (FIG. 4) in a non-Ca²⁺ dependent, but reversible fashion. No other PKC inhibitor had any effect on BK_{Ca} channel activity under basal conditions (not shown). Moreover, rottlerin's mechanism appears unique; tail currents are markedly prolonged after exposure to rottlerin, implying a slowing of deactivation, and the G-V curve is reversibly shifted by more than 100 mV to the left (FIG. 4B). Similar results were observed in a rat BK_{Ca} channel heterologously expressed in HEK293. Intracellular dialysis of rottlerin (the inventors tested up to 20 μM, which represents ˜200 fold more than the maximum extracellular concentration tested) had only a relatively small effect on the channel activity, suggesting that access to the activating site requires extracellular exposure (FIG. 5).

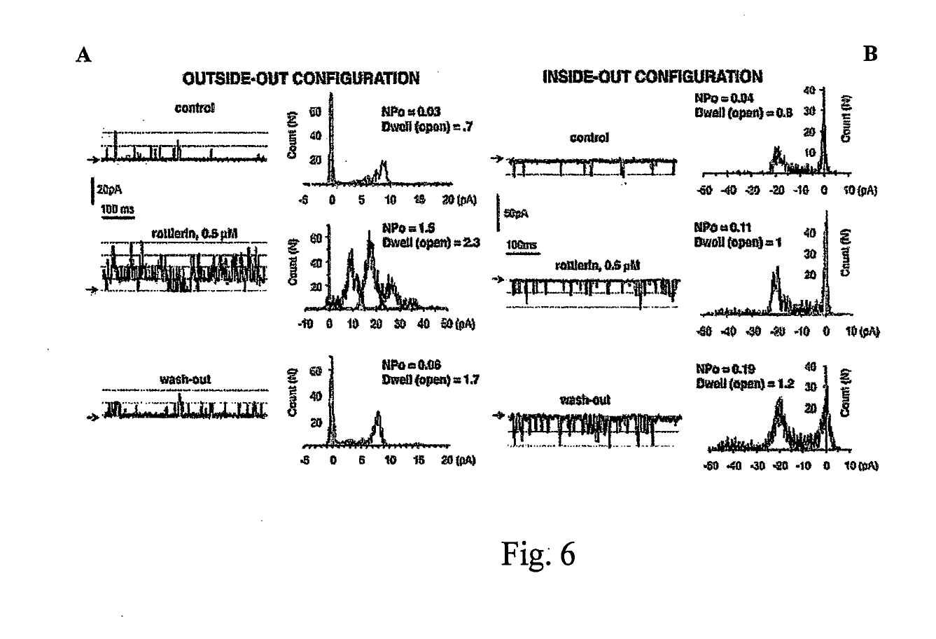

Although rottlerin has been proposed to have other effects at significantly high concentrations (˜10 μM), the BK_{Ca} activation is not due to modulation by PKC (or other cellular components), because it can be demonstrated using a cell-free configuration (FIG. 6).

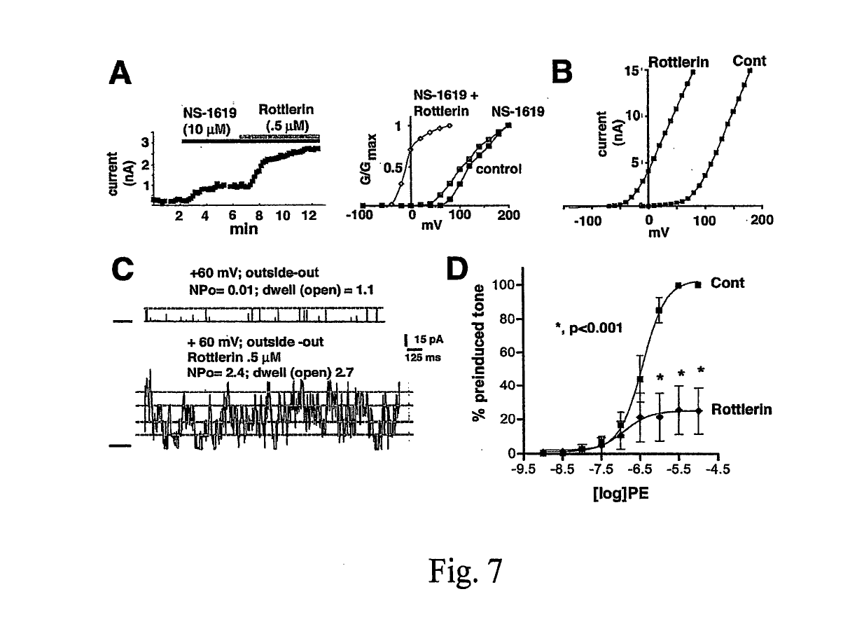

Rottlerin was compared to one of the originally described BK_{Ca} channel activators, NS-1619 (Olesen, et ah, Selective activation of Ca²⁺-dependent K⁺ channels by novel benzimidazolone. Eur. J. Pharmacol, 1994. 251(1):53-9). NS-1619 (10 μM) activated peak K⁺ current in HEK293 cells stably transfected with mSlo (FIG. 7A); addition of rottlerin (0.5 μM) after NS-1619 administration incrementally increased current in 0 Ca²⁺. Co-expression of the β1 subunit does not modify the effects of rottlerin. In HEK293 cells expressing both mSlo and β1 subunit, rottlerin activated the channel (FIG. 7B).

Given rottlerin's potent BK_{Ca} activating effects, in the absence of Ca²⁺ and the presence of the β1 subunit, the inventors hypothesized that rottlerin may be effective in mediating the relaxation of vascular smooth muscle. Human vascular smooth muscle cells (VSMC) grown in vitro express BK_{Ca} channels. Single channel recordings (outside-out configuration) demonstrate that rottlerin activated BK_{Ca} channels (FIG. 7C) by increasing Po and open dwell time.

Next, the inventors determined whether rottlerin could mediate vascular relaxation, as demonstrated for several other BK_{Ca} channel activators (Nardi, et al., Natural modulators of large-conductance calcium-activated potassium channels. Phnta. Med., 2003. 69(10):885-92). Rottlerin (4 μM) reduced phenylephrine mediated contraction by more than 50% (FIG. 7D), although 1 μM had no significant effect. The rottlerin mediated effect was inhibited by TEA, suggesting a prominent K⁺ channel contribution to the blunting of contractile tone. The difference in concentration between the BK_{Ca} channel effects in electrophysiologic experiments compared to vascular rings may be explained by the hydrophobic properties of the compound and the experimental conditions. Interestingly, NS-1619's efficacy in mediating vascular relaxation was diminished in a hypertensive rat model (Callera, et al., Ca²⁺-activated K⁺ channels underlying the impaired acetylcholine-induced vasodilation in 2K-1C hypertensive rats. J. Pharmacol. Exp. Ther., 2004. 309(3): 1036-42), compared to controls, perhaps consistent with the down-regulation of the β1, but not α subunit observed in hypertensive animal models (Amberg, et al., Downregulation of the BK channel β1 subunit in genetic hypertension. Circ. Res., 2003. 93(10):965-71; Amberg, et al., Modulation of the molecular composition of large conductance, Ca²⁺ activated K⁺ channels in vascular smooth muscle during hypertension. J. Clin. Invest., 2003. 112(5):717-24). Based upon rottlerin's efficacy in in vitro electrophysiologic experiments (FIG. 4-7), in low [Ca²⁺]ᵢ and in the absence of a β1 subunit, the inventors hypothesize that rottlerin-like compounds may be more effective.

### Example 2

Rottlerin-Induced Activation of BK Channels does not Involve Phosphorylation or Cytosolic Components

Murine tracheal smooth muscle cells were isolated using the following protocol. Trachea were removed, cut longitudinally, the epithelium removed (brushing with sterile cotton bud) and the cartilage removed by cutting. The isolated trachea were dissected in culture medium and cut into several pieces (1-2 mm²). After addition of 0.5 mg/ml papain (Roche, Nutley, N.J.) and 1 mg/ml dithiothreitol, the cells were dissociated at 37° C. for 20 minutes, gently shaking, followed by the addition of 0.1 mg/ml liberase enzyme 2 (Roche, Nutley, N.J.) for 30 minutes at 37° C. (5% CO₂). The suspension was then pipetted gently several times to disburse cells, the cell suspension strained via a nylon cell strainer, and the suspension was gently triturated to disburse single cells. The cell suspension was then centrifuged at 700×g for 5 minutes and the pellet resuspended in 500 μl Krebs solution. The cells were plated onto laminin-coated tissue culture dishes. BK currents were recorded using whole-cell and outside-out macropatches.

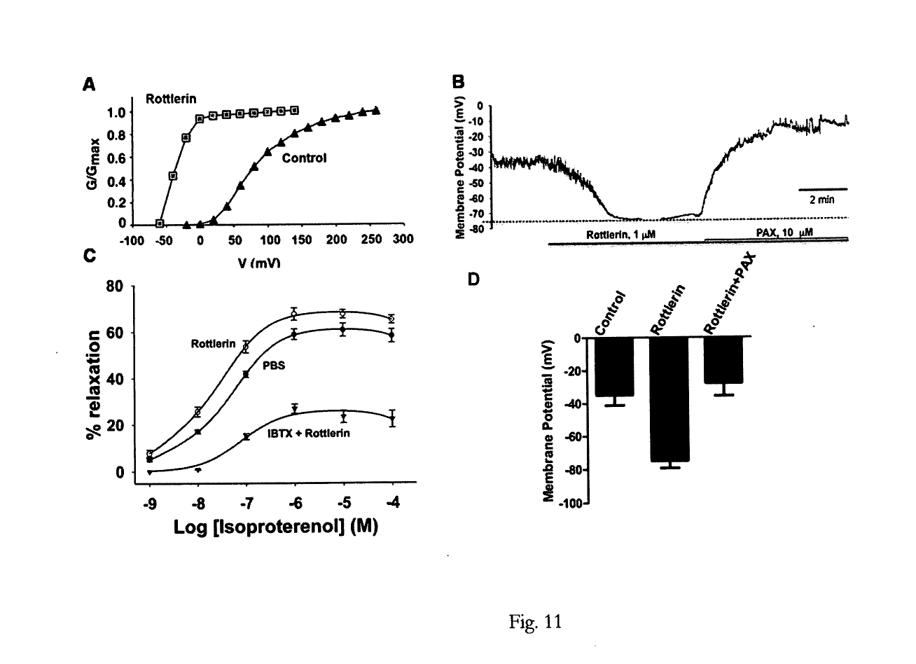

As shown in FIG. 11A, rottlerin (1 mM) significantly activated BK currents in tracheal smooth muscle. When measuring the membrane potential, a significant hyperpolarization of the membrane was observed after rottlerin application, which was completely reversed after administration of paxilline, a BK channel antagonist (FIGS. 11B and 11D).

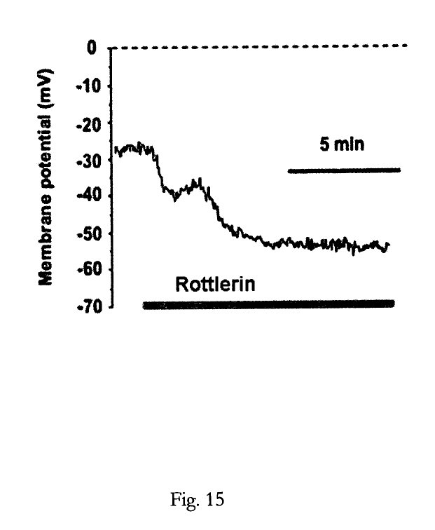

Rottlerin's ability to induce hyperpolarization of the membrane potential of tracheal smooth muscle cells was confirmed by another set of experiments. The membrane potential was recorded using perforated patch clamp technique. As shown in FIG. 15, rottlerin induced significant hyperpolarization of the membrane potential of tracheal smooth muscle.

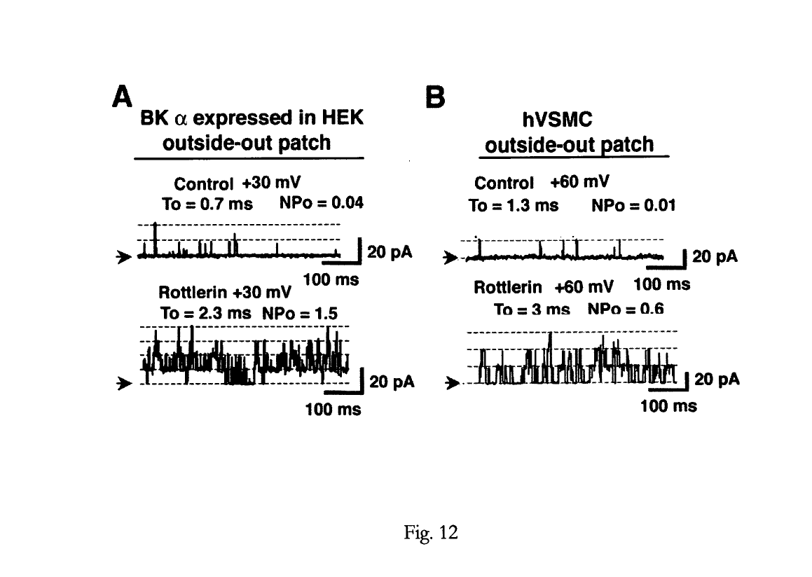

In outside-out patches pulled from HEK cells stably expressing BK channels and in cultured human VSMC, bath application of 0.5 μM rottlerin resulted in a significant increase in channel open probability (FIG. 12), which was reversed with wash-out (not shown). The reversible activation of BK channels by rottlerin in a cell-free configuration, in the absence of ATP, implies that rottlerin-induced activation of BK channels does not involve phosphorylation or cytosolic components.

### Example 3

Isoproterenol-Induced Relaxation was Dependent Upon BK Channel-Induced Hyperpolarization

It is well-known that β-adrenergic agonists promote relaxation of airway smooth muscle. Inhibition of BK channels has been shown to reduce β-adrenergic agonist-induced relaxation.

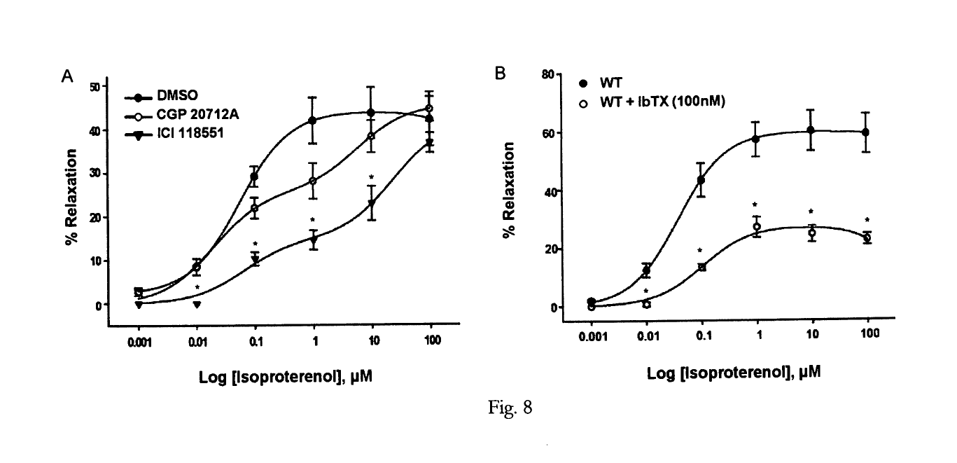

To dissect which (or both) β-AR pathways are responsible for the effects, tracheal rings were pre-incubated with either β1-AR antagonist (CGP 20712A; 100 nM) or a β2-AR antagonist (ICI 118551; 100 nM) or vehicle (DMSO). FIG. 8A shows that pre-incubation of rings with β2-AR antagonist resulted in a rightward shift in the dose-response curve indicating that β2AR pathway is primarily responsible for the relaxation. To confirm that the isoproterenol-induced relaxation was dependent upon BK channel-induced hyperpolarization, IbTX, a specific inhibitor of the BK channel, was used. FIG. 8B shows that in the presence of IbTX, tracheal rings of WT showed a significant decrease in relaxation in response to isoproterenol, confirming prior published results.

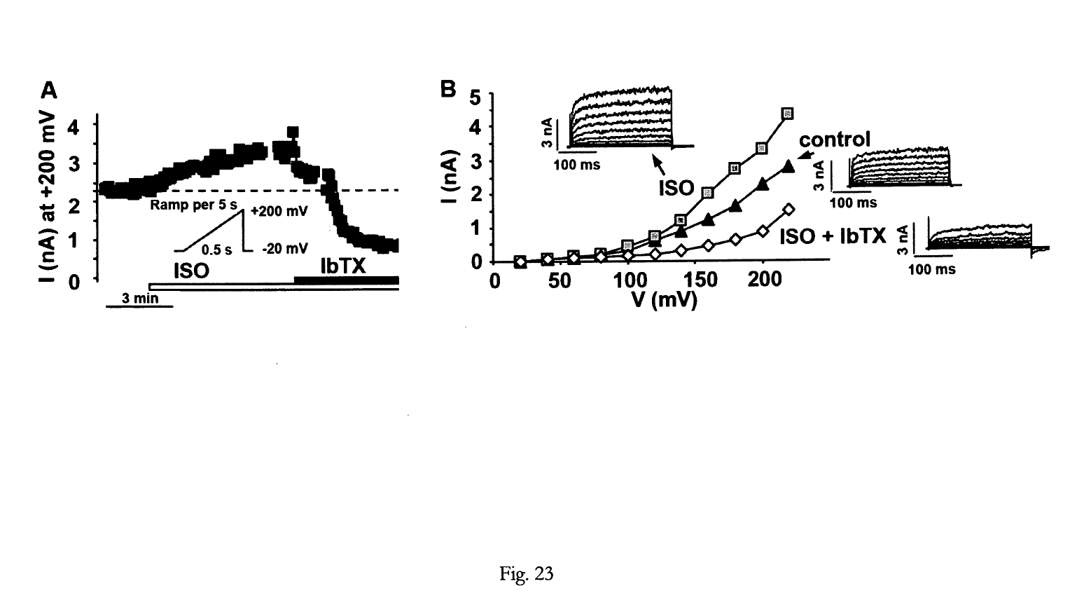

The effect of ISO on BK currents in acutely isolated tracheal ASM was also determined. ASM cells were studied using perforated whole cell voltage clamp. After obtaining access, outward K⁺ current was monitored using a 0.5 second ramp, from a holding potential of −20 mV to +200 mV. After recording a stable baseline, the cells were exposed to 0.5 μM ISO, which increased outward K⁺ current by more than 50%, which was substantially blocked by IbTX (FIGS. 23A and 23B). Under the conditions used, the majority of the outward K⁺ current is conducted by the BK channel, as shown by IbTX-blockade (FIGS. 23A and 23B).

To test the hypothesis that rottlerin-induced hyperpolarization causes relaxation of ASM, rottlerin was added on tracheal rings mounted on a myograph, and ISO-induced relaxation was measured. Rottlerin enhanced the ISO-induced relaxation of tracheal rings when compared to PBS, shifting the ISO-relaxation curve upward and to the left (FIG. 11C). However, tracheal rings pre-incubated with iberiotoxin (IbTx), a BK channel inhibitor, showed minimal relaxation to ISO, even in the presence of rottlerin further indicating rottlerin's effect via BK channels.

### Example 4

### Administration of Rottlerin in Asthma Model

In this example, the acute asthma model in ovalbumin (OVA) treated mice was used (Jia, Y., R. Foronjy, and J. D'Armiento, Altered airway inflammatory response to cigarette smoke in ovalbumin-sensitized mice. Am J. Resp and Critical Care Medicine (2006) p. Suppl:A339.). Rottlerin was administered to asthmatic mice to test whether it attenuated the development of asthma. Mice received an intraperitoneal injection (i.p) of 100 μg ovalbumin adsorbed to 2 mg aluminum (200 μl final volume) on day 0 and again on day 7. Control mice were injected with endotoxin free PBS. On alternative days 14-22, mice received a 20 minute aerosol challenge of either endotoxin-free PBS (controls) or 2% (w/v) OVA in endotoxin-PBS, using a lumiscope 6610 ultrasonic nebulizer (Lumiscope, East Rutherford, N.J.). For rottlerin (Sigma, St. Louis, Mo.) treatment mice received one i.p injection of 5 mg per kg body weight on day 13, one injection an hour before each aerosol challenge and one before methacholine challenge.

Two days after the last aerosol challenge, AHR was measured by invasive restrained whole body plethysmography (rWBP) (BUXCO Electronics Inc., Troy, N.Y.) in response to inhaled methacholine (Sigma, St. Louis, Mo.). For dynamic lung resistance, measurements were performed using the PLY3011 chamber (BUXCO). Mice were anesthetized with a cocktail of 25 mg per kg body weight ketamine hydrochloride (Bioniche Pharma, Lake Forest, Ill.) and 2.5 mg per kg body weight xylazine (Llyod Laboratories, Inc., Barangay Tikay, Malolos Bulacan, Philippines), tracheotomized, and immediately intubated with an 18-gauge catheter, followed by mechanical ventilation (Columbus Instruments International, Columbus, Ohio). Respiratory frequency was set at 150 breaths/minute with a tidal volume of 0.2 ml. Increasing concentrations of methacholine (0-50 mg/ml) were administered at the rate of 20 puffs per 10 seconds, with each puff of aerosol delivery lasting 10 ms, via a nebulizer aerosol system with a 4-6 μm aerosol particle size generated by a nebulizer head (Aeroneb®, Aerogen Ltd., Dangan, Galway, Ireland). The nebulizer is attached to a small aerosol block, which is placed in the inspiratory flow line. In this way the aerosol is injected directly into the airway. Baseline resistance was restored before administration of the subsequent doses of methacholine. The flow and pressure signals were measured and processed together to determine resistance and compliance using a software analyzer provided in BioSystem XA software (BUXCO Electronics Inc., Troy, N.Y.).

After measurement of airway responsiveness in vivo, mice were sacrificed and their serum collected and stored at −80° C. until analysis. Serum levels of OVA-specific IgE were measured by sandwich ELISA as described previously (Kanamaru, F., et al. “Costimulation via Glucocorticoid-Induced TNF Receptor in Both Conventional and CD25⁺ Regulatory CD4⁺ T Cells.” J Immunol., Vol. 172, pages 7306-7314 (2004)).

Bronchoalveolar lavage (BAL) was performed by injection of 1 ml saline (37° C.) through a tracheal cannula into the lung. Cells in the BAL were centrifuged and resuspended in cold PBS. For differential BAL cell counts, cytospin preparations were made and per cytospin, 200 cells were counted and differentiated by standard morphology and staining characteristics.

IL-4, IL-5 and IL-13 ELISAs were performed according to the manufacturer's instructions (R & D systems, Minneapolis, Minn.). The detection limits of the ELISAs were 60 pg/ml for IL-4, 32 pg/ml for IL-5, 15 pg/ml IL-13.

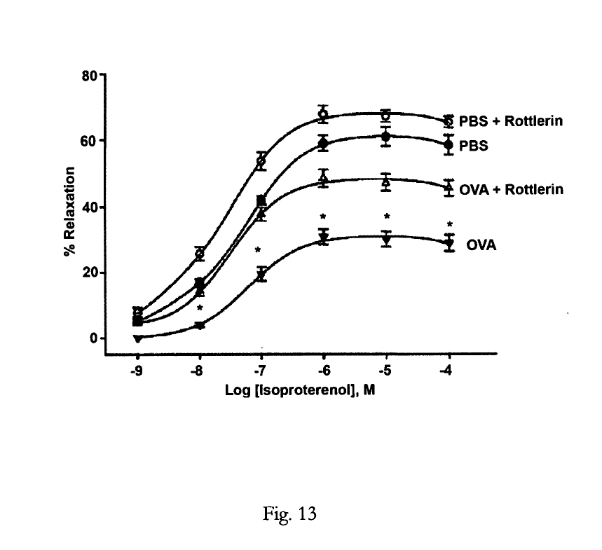

The isoproterenol-induced relaxation of tracheal rings from OVA-sensitized asthmatic animals was also tested. After induction of asthma, tracheal rings were removed and mounted on the myograph. Rottlerin (0.5 mM) was added to the bath of each tracheal ring 2 minutes prior to exposure to isoproterenol. At the conclusion of the experiment, each tracheal ring was exposed to methacholine to ensure viability of the ring and equivalent constriction. Rottlerin enhanced the isoproterenol-induced relaxation of the PBS-treated (control) animals, shifting the isoproterenol-relaxation curve upward and to the left (FIG. 13). This result indicates that rottlerin can induce relaxation of tracheal rings in a basal state (not exposed to methacholine), indicating that there is at least an additive effect of rottlerin and isoproterenol.

Trachea obtained from OVA-sensitized animals had a blunted relaxation response to isoproterenol (FIG. 13). The isoproterenol-relaxation curve was shifted downward and to the right, with the maximal relaxation response reduced to only ˜25% from more than 60%. Remarkably, acute exposure to rottlerin restored the response to ˜45%, and caused a leftward shift in the isoproteronol-relaxation curve to near-normal. These results indicate that rottlerin has a direct and an acute effect on airway hyper-responsiveness in vitro and these results laid the foundation for administering rottlerin as a therapeutic for asthma.

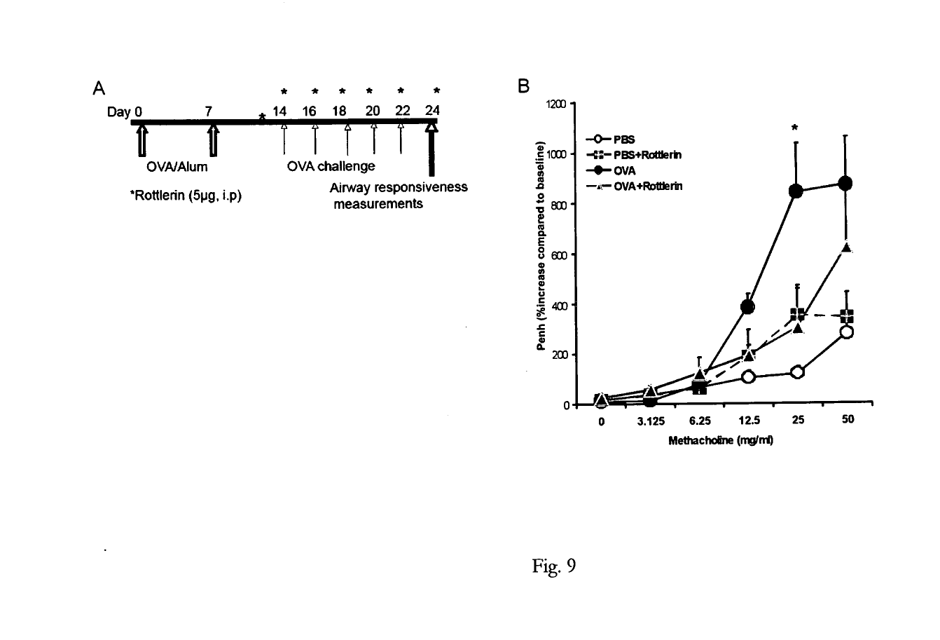

FIG. 9B shows the increase in airway hyper-responsiveness in response to increasing doses of MCh. As expected, in response to 25 mg/ml MCh, OVA-treated mice exhibited increased bronchoconstriction compared to the PBS sensitized mice. Rottlerin-treated mice sensitized with OVA showed a decrease in airway constriction to OVA-treated animals at 25 mg/ml methacholine challenge.

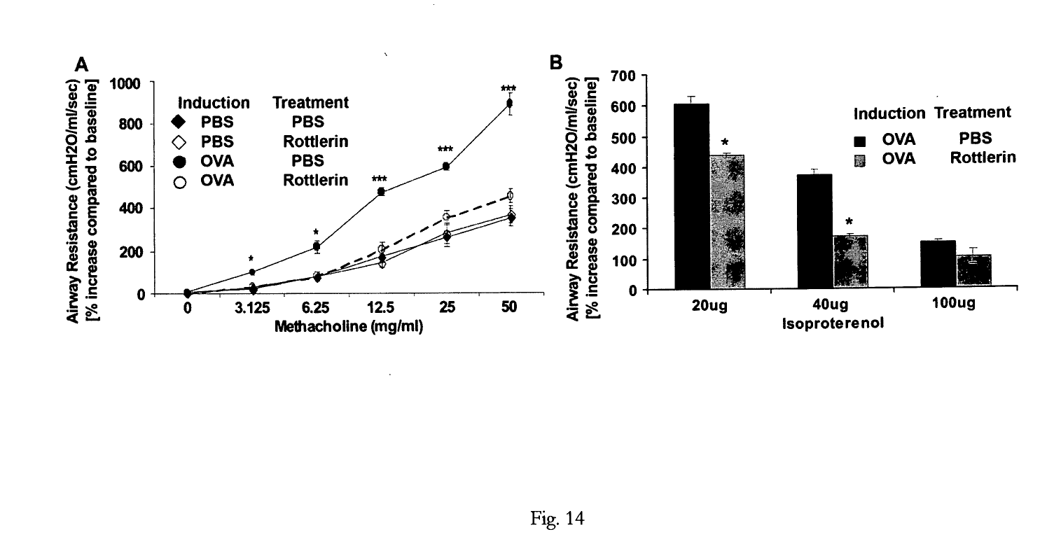

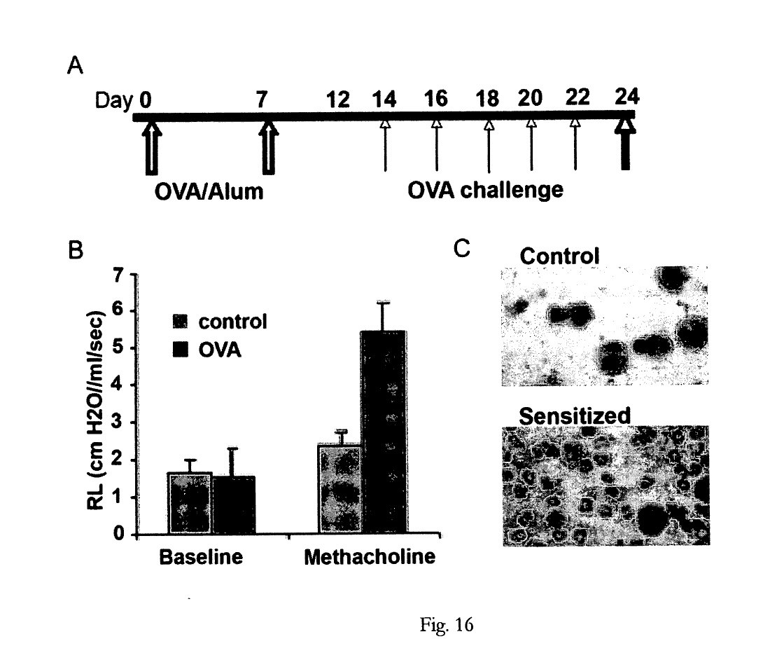

To further confirm these results, airway resistance (RL) in these animals was measured. In this system mice are anesthetized and ventilated and through a cannula in the trachea, tracheal pressure and flow are continuously monitored and traditional pulmonary mechanics can be measured. This system is preferable over the method of oscillatory mechanics due to the consistency of measurements and the ability to directly measure pressure and flow. Changes in pressure, flow, and volume were recorded, and RL was calculated from peak values after each challenge (FIG. 14A). Administration of rottlerin had no significant adverse effect on any treated mouse—there was no apparent morbidity or any mortality. Rottlerin-treatment of PBS-sensitized animals had no effect on airway resistance (FIG. 14A). Indeed, the OVA-sensitized mice showed an increase in their RL as compared to the PBS-sensitized mice in response to Mch (25-50 mg/ml). Rottlerin treated mice exhibited a decrease in their airway resistance (FIG. 14A and FIG. 16B). These results indicate that rottlerin may play a therapeutic role in attenuating or preventing the development of asthma.

To further understand the role of rottlerin, increasing doses of isoproterenol (i.p. 20, 40 and 100 μg) were administered to the OVA-sensitized mice (PBS or rottlerin treated) after the last methacholine-challenge of 50 mg/ml. Rottlerin treated OVA-sensitized mice showed a significant decrease in airway constriction as compared to the untreated group, suggesting an additive effect of rottlerin with isoproterenol (FIG. 14B).

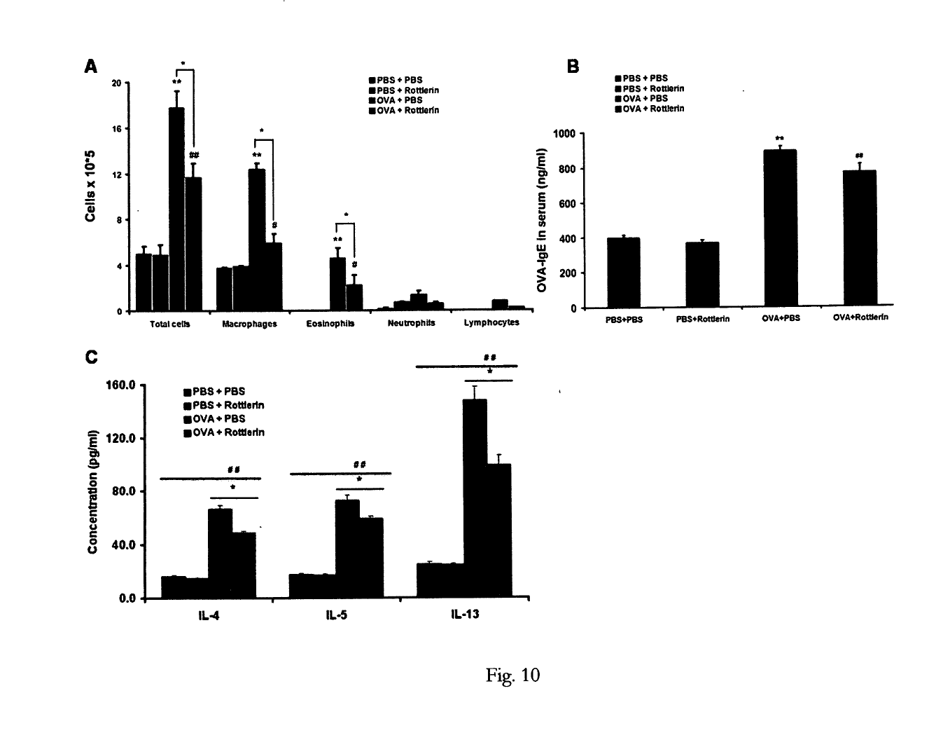

After determining the airway hyperresponsiveness, lungs were lavaged with PBS and total cell count with differential was determined on the bronchoalveolar cells (BAL). Cytospin was performed on the collected lavage, and cells were stained with Diff Quik (IMEB, Inc., San Marcos, Calif.). Cell type (e.g., eosinophils, marcrophages and lymphocytes) were recognized by morphometry. The OVA-sensitized mice exhibited an increase in inflammation and differential count (eosinophils, lymphocytes and macrophages) (FIG. 16C) (Wang et al., “Endogenous and exogenous IL-6 inhibit aeroallergen-induced Th2 inflammation” J Immunol 165:4051-4061 (2000)). FIG. 10A shows inflammatory leukocytes recruited into the lungs following sensitization and challenge with OVA. OVA-sensitized mice showed an increase in the number and variety of cells, including macrophages, eosinophils, and neutrophils as compared to the unsensitized groups. The OVA-sensitized mice treated with rottlerin showed a significant decrease in the number of inflammatory leukocytes with a marked reduction in the number of macrophages and eosinophils. These results suggest that rottlerin plays an important role in preventing the inflammatory response in asthma. To confirm sensitivity with OVA, specific IgE levels were examined 48 hours after the last airway challenge. Following systemic OVA sensitization and challenge, there was a significant increase in the serum IgE levels in the OVA sensitized groups (OVA; OVA+rottlerin) (FIG. 10B). The serum IgE in the OVA and OVA+rottlerin groups were similar demonstrating that the animals were similarly sensitized. The serum IgE levels in both these groups were approximately equivalent, demonstrating that the animals were equally sensitized. Consistent with the observed changes in R_{L} in response to MCh and the reduction in inflammatory cells in the BALF in response to rottlerin, a significant reduction in the Th2 cytokine production was observed in the BALF of the rottlerin-treated animals. All Th2 cytokines evaluated, IL-4, IL-5, and IL-13, were significantly increased in the BALF of the OVA-sensitized animals (FIG. 10C). However, the production of all these Th2 cytokines was significantly reduced in the BALF of rottlerin-treated OVA-sensitized animals. There was no significant difference between the PBS- and rottlerin-treated control groups.

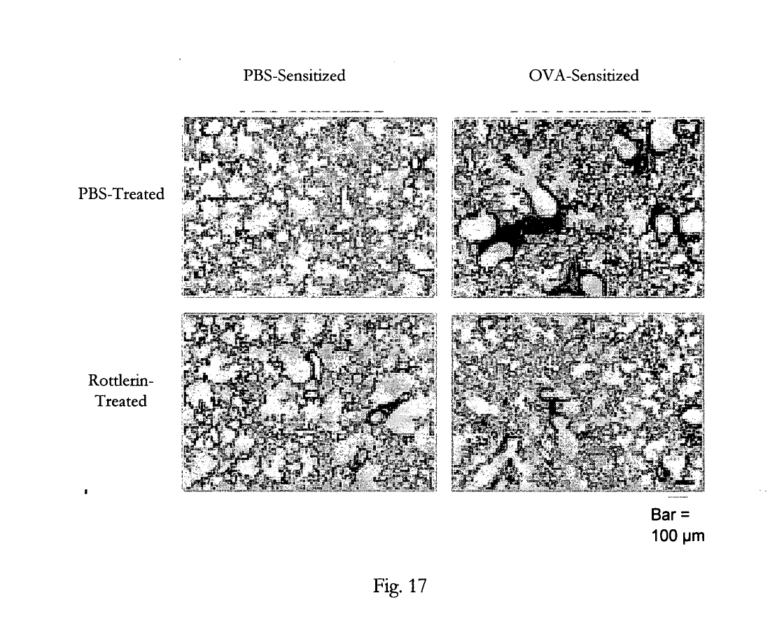

Consistent with the observed changes in airway resistance in response to methacholine and the reduction in inflammatory cells in the BAL fluid in response to rottlerin, a significant reduction in the cellular infiltrate in the peribronchial and perivascular regions was observed in rottlerin-treated OVA-sensitized/challenged animals compared to PBS-treated OVA-sensitized/challenged animals (FIG. 17).

Thus, in the acute asthma model, rottlerin attenuated the OVA-induced airway hyper-reactivity and pulmonary resistance and reduced inflammation, demonstrating that rottlerin may play an important therapeutic role in preventing or attenuating the development of airway hyperreactivity.

### Example 5

### Single Dose of Rottlerin Acutely Relaxes OVA-Induced AHR

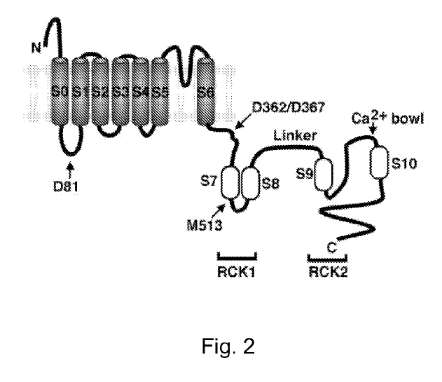

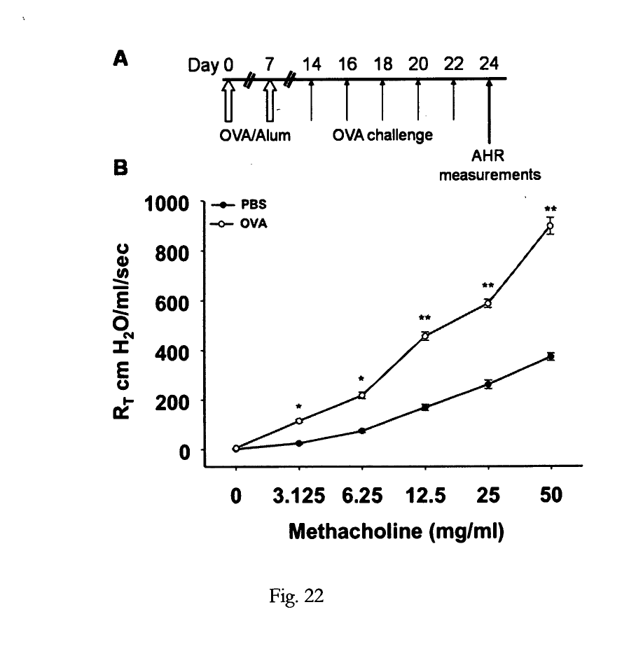

Based upon the proposed role for BK channels in modulating airway contractility, the inventors hypothesized that acute administration of rottlerin, like β2 adrenergic agonists, can cause bronchodilatation. An acute asthma model in OVA-sensitized mice established and validated, as assessed by measuring airway resistance (R_{L}) in response to methacholine (MCh). Groups of mice received an I.P. injection of OVA/Alum complex on days 0 and 7 and on alternate days 14-22, a 20 minute aerosol challenge of either PBS or 2% (w/v) OVA in PBS, using an ultrasonic nebulizer (FIG. 22A). The asthma model exhibited an increase in AHR as shown by an increase in R_{L} in response to MCh (FIG. 22B). To determine whether rottlerin could reverse AHR in OVA-asthmatic mice, a single dose of rottlerin (5 μg/g) was injected intravenously (I.V.) 5 minutes before measurements of AHR (FIG. 2A). As expected, the OVA-sensitized mice exhibited an increase in R_{L} as compared to controls in response to MCh (0-50 mg/ml). However, the OVA-sensitized group that received rottlerin I.V. showed a significant decrease in their R_{L} when compared to the untreated groups (FIG. 2B). These results indicate that acute treatment of rottlerin can reverse OVA-induced AHR.

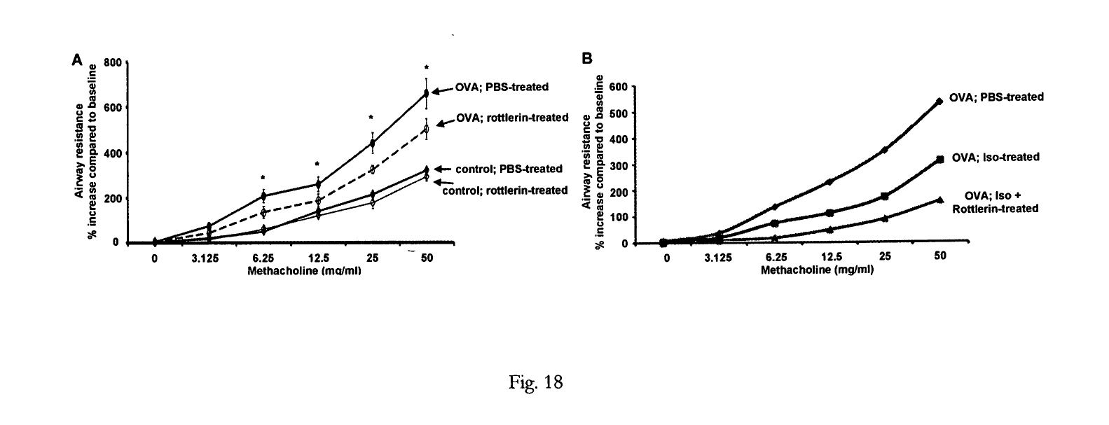

Control and OVA-challenged mice were treated with rottlerin, given through the tail vein 5 minutes prior to airway pressure measurements. Rottlerin significantly reduced airway resistance in the OVA-sensitized/challenged animals (FIG. 18A), compared to PBS-treated OVA sensitized/challenged animals. This acute effect is unlikely to be due to an effect on inflammation given the short period of time between injection of the drug and measurement of airway resistance, strongly suggesting a direct effect of rottlerin on smooth muscle contractility, likely by activating BK channels and hyperpolarizing membrane potential. Acute administration of rottlerin, in combination with Isoproterenol, increased β-agonist mediated relaxation of the airway in OVA-challenged asthmatic mice (FIG. 18B).

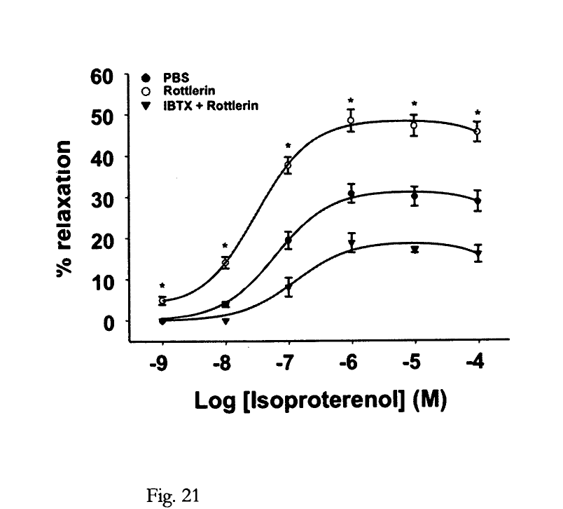

Next, whether rottlerin can restore isoproterenol (ISO)-induced relaxation of tracheal rings in OVA-sensitized asthmatic animals was tested. After induction of asthma, tracheal rings were removed and mounted on the myograph. Rottlerin (0.5 μM) was added to the bath of each tracheal ring 2 minutes prior to exposure to ISO. As seen in FIG. 21B, tracheas obtained from OVA-sensitized animals had a blunted relaxation response to ISO. The ISO-relaxation curve was shifted downward and to the right, with the maximal relaxation response reduced to only ˜25% from more than 60%. Remarkably, acute exposure to rottlerin in the bath restored the response to ˜45%, and caused a leftward shift in the ISO-relaxation curve to near-normal. These results indicate that rottlerin has a direct and an acute effect on airway hyper-responsiveness in vitro.

### Example 6

### House Dust Mite Antigen Induction of Asthma

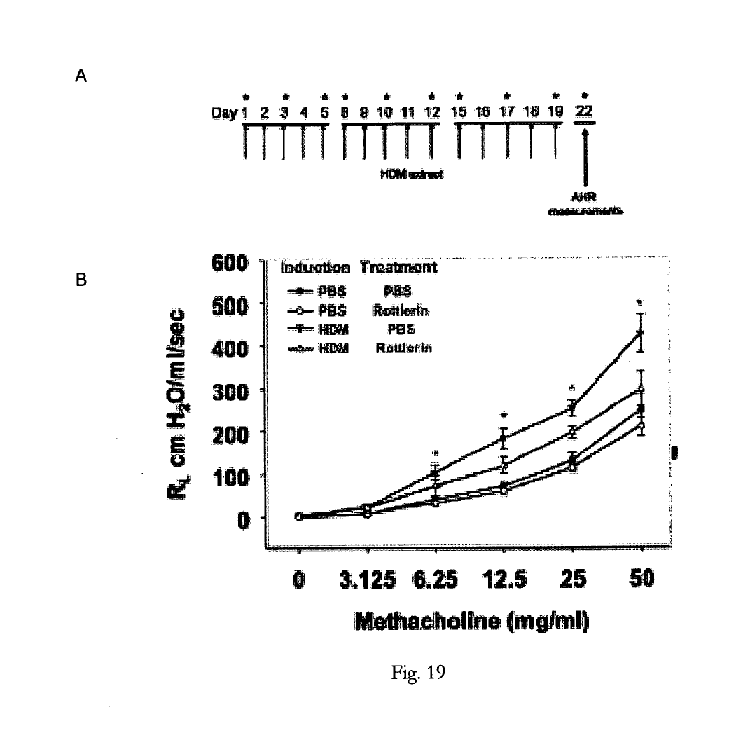

Whether rottlerin could affect AHR in the house dust mice antigen model of asthma was determined. House dust mite (HDM) is one of the most common aeroallergens and is implicated in allergy and asthma symptoms in ˜10% of the population (Johnson et al., “Continuous exposure to house dust mite elicits chronic airway inflammation and structural remodeling.” Am J Respir Crit Care Med 169:378-385 (2004)). Exposure to HDM extract elicits a severe and persistent eosinophilic airway inflammation. Mice were exposed to purified HDM extract (Greer Laboratories, Lenoir, N.C.) intranasally (25 μg of protein in 10 μl saline) for 5 days/week for 3 weeks as previously described (Id.). Rottlerin (5 μg/g=100 μg/mouse intraperitoneal) was administered every other day (see protocol in FIG. 19A). Changes in pressure, flow, and volume were recorded, and airway resistance was calculated from peak values after each challenge using a Buxco forced maneuvers system and restrained whole body plethysmography (rWBP; PLY3011 chamber, Buxco (BUXCO Electronics Inc., Troy, N.Y.)). Administration of rottlerin had no significant adverse effect on any treated mouse—there was no increase in mortality or apparent morbidity in rottlerin-treated animals. Rottlerin-treatment of PBS-sensitized animals had no effect on airway resistance

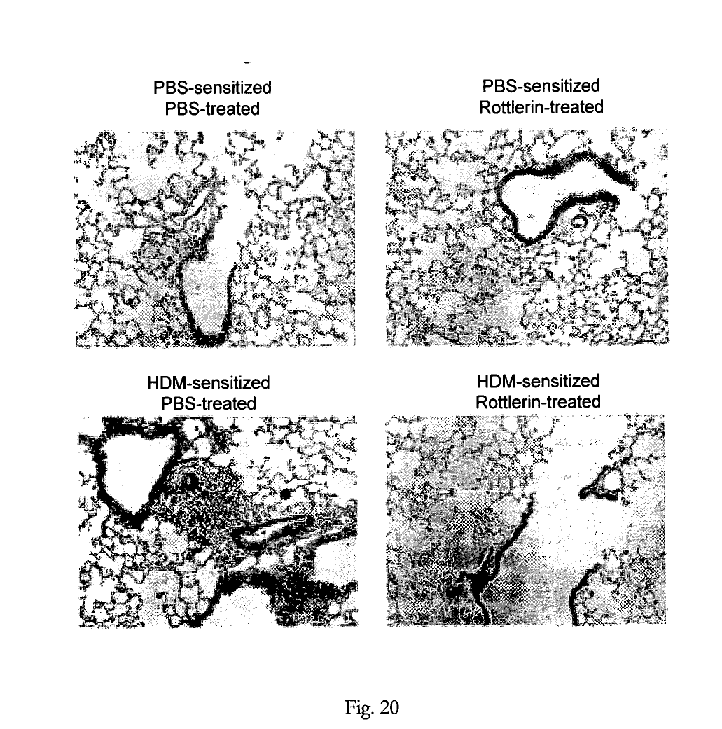

As expected, the HDM-exposed mice exhibited an increase in airway resistance as compared to PBS-sensitized mice in response to methacholine (25-50 mg/ml) (FIG. 19B). The rottlerin-treated, HDM-sensitized mice exhibited a marked decrease in their airway resistance when compared to HDM-sensitized, PBS-treated animals (FIG. 19B). Consistent with the observed changes in airway resistance in response to methacholine, a significant reduction in the cellular infiltrate was observed in the peribronchial and perivascular regions in rottlerin-treated HDM-exposed animals compared to PBS-treated HDM-exposed animals (FIG. 20).

The predominant features of asthma are: A) the inappropriate constriction of airway smooth muscle (ASM), and B) inflammation. The contractility of airway smooth muscle is regulated in part by plasma membrane BK channels (large conductance voltage- and Ca²⁺-activated K⁺ channels). BK channel activation causes transient membrane hyperpolarization, inhibition of Ca²⁺ influx through voltage-dependent Ca²⁺ channels, reduced [Ca²⁺]ᵢ and smooth muscle relaxation. Evidence supporting a role for BK channels in modulating airway contractility is based upon animal and human studies in which reduction in BK channel function is associated with airway hypercontractility. Moreover, polymorphisms in the BK channel have been identified that are associated with more severe forms of asthma in humans. The inventors have shown that a small molecule, rottlerin, directly and potently activates BK channels. Systemic administration of rottlerin, both acutely and chronically, attenuates the induction of airway hyperreactivity in two models of asthma: (1) ovalbumin exposure model; (2) house dust mice exposure model. In addition, the inventors showed that rottlerin potently inhibits the immunological response, with a marked reduction in peribronchial and perivascular infiltration of immune cells. Moreover, the inventors demonstrated that a single injection of rottlerin via the tail vein of mice can significantly diminish methacholine-induced airway hyperreactivity in the ovalbumin asthma model. The effects on reducing airway hyperreactivity are likely mediated both through BK channel-dependent effects on smooth muscle relaxation and through BK channel-independent effects on immunologic mediators of asthma. By targeting BK channels, which are not expressed in the heart, airway smooth muscle reactivity may be normalized without the side-effects observed with standard therapies. This approach is entirely novel as it combines anti-inflammatory actions with direct airway smooth muscle relaxation in a single therapeutic.

### Example 7

### Dosage Determination and Aerosolized Delivery of Rottlerin

The data above demonstrate that systemic (I.P.) administration of rottlerin for 2 weeks during OVA or HDM challenge markedly attenuates AHR, and inflammation in OVA and HDM sensitized mice. Whether aerosolized delivery of the drug is effective in these two asthma models will be tested. Aerosolized delivery has the advantages of: 1) direct delivery to the target organ which has a large absorptive surface and eliminates first pass metabolic degradation; 2) potentially reducing systemic adverse effects (e.g. lowering BP); 3) rapid onset of action. For these reasons most currently effective therapies for asthma including steroids, and bronchodilators are delivered using aerosolized forms (Sears et al., “Regular inhaled beta-agonist treatment in bronchial asthma.” The Lancet, 1990. 336(8728): p. 1391-1396; Barnes, P. J., “Inhaled glucocorticoids for asthma.” N Engl J Med, 1995. vol. 332(13): p. 868-75.)

Ultrasonic nebulization for the airway delivery of rottlerin will be used as described (Hrvacic et al., “Applicability of an ultrasonic nebulization system for the airways delivery of beclomethasone dipropionate in a murine model of asthma.” Pharm Res, 2006. 23(8): p. 1765-75; Wiedmann, T. S. and A. Ravichandran, Ultrasonic nebulization system for respiratory drug delivery. Pharm Dev Technol, 2001. 6(1): p. 83-9). Ultrasonic nebulizers produce aerosols from drug solutions by converting electrical pulses to mechanical vibrations (Atkins, P. and A. R. Clark, Drug delivery to the respiratory tract and drug dosimetry. J Aerosol Med, 1994. 7(1): p. 33-8). These nebulizers have been previously used for the inhalation delivery of anti-asthma drugs in murine models of asthma (Hrvacic et al., “Applicability of an ultrasonic nebulization system for the airways delivery of beclomethasone dipropionate in a murine model of asthma.” Pharm Res, 2006. 23(8): p. 1765-75). Microsuspensions of rottlerin will be aerosolized using a nebulizer and delivered to mice via a nose-only inhalation route; the total dose will be controlled by varying formulation strength while holding exposure time constant.

To determine basic pharmacokinetic profiles, mice will be exposed to aerosols for a single 30-minute period; then, 30 minutes post-treatment, lung tissues, plasma, and BALF will be analyzed for rottlerin content. The aerosolized rottlerin will be administered for 5 or 21 days and lung tissues, plasma, and BALF will be analyzed for rottlerin content to determine whether steady state levels are achieved and what the tissue half-life [t½] of rottlerin is. To correlate these exposure levels with safety, animals will be monitored for changes in blood pressure, weight, and heart rate. Relevant organs including lungs, heart, liver, kidneys, and digestive tract will be examined for histological changes after treatment compared with organs taken from untreated mice. Aerosolized rottlerin (molecular formula: C₃₀H₂₈O₈, MW: 516) will be prepared according to published protocols used for compounds of similar molecular weight and solubility. Rottlerin is soluble to 2 mM in ethanol and to 100 mM in DMSO. The ultrasonic nebulization system (UNS) that will be used consists of an ultrasonic nebulizer, drying column, deionizer and animal exposure chamber. Solutions will be pumped into the nebulizer with a syringe pump. The nebulizer and an ultrasonic spray nozzle system will be driven by an ultrasonic generator operating at a fixed frequency of 125 kHz attached to an air supply device that injects a stream of air (3 l/min) around nozzle of the nebulizer mounted on the upper conical portion of a 500 ml round-bottom flask connected to a 45 cm long drying column using tygon tubing. Spray drying of the aerosol is accomplished by passage through the inner cylinder of drying column surrounded by charcoal. The outlet of the drying column is connected through the deionizer to an animal exposure chamber. Using the established asthma protocols, OVA, HDM and control (PBS) mice will receive a 20 minute aerosol treatment with either DMSO (0.1% in PBS) or rottlerin (0.1, 1, 10 and 100 μg/g in a total volume of 150 μl PBS) the dose administered to the mice will be estimated according to the formula described (Wattenberg et al., “Chemoprevention of pulmonary carcinogenesis by brief exposures to aerosolized budesonide or beclomethasone dipropionate and by the combination of aerosolized budesonide and dietary myo-inositol” Carcinogenesis, vol. 21(2): p. 179-82 (2000)), using a lumiscope 6610 ultrasonic nebulizer (Lumiscope Co. Inc., Piscataway, N.J.). Mice will be intubated and R_{L} measured before and after MCh challenge. The inflammatory components of asthma (inflammatory cells and perivascular/peribronchial cellular infiltration) will be assessed for the untreated and rottlerin-treated asthmatic animals as described in the preliminary data section. The following will be determined: 1) the dose-response curve for rottlerin and calculate the half maximal effective concentration (EC₅₀); 2) the effects on blood pressure since BK channels are present in vascular smooth muscle (Brenner, R., G. J. Perez, A. D. Bonev, D. M. Eckman, J. C. Kosek, S. W. Wiler, A. J. Patterson, M. T. Nelson, and R. W. Aldrich, “Vasoregulation by the beta1 subunit of the calcium-activated potassium channel” Nature, vol. 407(6806): p. 870-6 (2000); Ledoux, J., M. E. Werner, J. E. Brayden, and M. T. Nelson, “Calcium-activated potassium channels and the regulation of vascular tone” Physiology (Bethesda), Vol. 21: p. 69-78 (2006)); 3) whether mucin production is reduced by rottlerin (via mucin staining of histological sections) as has been reported (Park et al., “Protein kinase C delta regulates airway mucin secretion via phosphorylation of MARCKS protein” Am J Pathol. Vol. 171(6): p. 1822-30 (2007)); and 4) the minimal effective dose in OVA and HDM asthma models. The R_{L} measured in response to MCh (12.5 mg/ml) will be plotted against the rottlerin dose to obtain the dose-response curve which will yield: a) potency, b) maximal efficacy or ceiling effect (greatest attainable response), c) slope (change in response per unit dose), d) EC₅₀ (half maximal effective concentration), e) minimal effective dose, f) maximal tolerated dose. These studies will provide the basis for estimating pharmacokinetics of rottlerin and the design of clinical trials.

As an alternative to aerosolized rottlerin, the use of liposome mediated delivery will be explored. Liposome mediated delivery has been used successfully to deliver drugs with similar characteristics to rottlerin to the lungs (Chougule, M., B. Padhi, and A. Misra, “Nano-liposomal dry powder inhaler of tacrolimus: preparation, characterization, and pulmonary pharmacokinetics” Int J Nanomedicine, Vol. 2(4): p. 675-88 (2007); Waldrep, J. C., “New aerosol drug delivery systems for the treatment of immune-mediated pulmonary diseases” Drugs Today (Barc), Vol. 34(6): p. 549-61 (1998)).

To determine whether rottlerin administration after asthma is established is effective and determine the duration of action of a single rottlerin administration in two murine asthma models, a dosing schedule will be used. In this dosing schedule, rottlerin will be administered every other day, starting on day 12 of a 24-day asthma-induction protocol, just prior to the OVA or HDM nebulization. A final dose of rottlerin will be given 1 hour prior to the assessment of AHR on the final day of the asthma induction protocol. Moreover, the examples above have shown that a single I.V. administration of rottlerin can acutely (within 5 minutes) reduce AHR in these models. The goal is to determine whether rottlerin administered every other day for 1-3 weeks after asthma has been established attenuates both the inflammatory response and AHR in the OVA-induced and HDM models of asthma. The effects of rottlerin on airway remodeling in the HDM model will also be examined. In addition, the duration of action of a single administration of aerosolized rottlerin will be determined.

The details of the dosage schedule for rottlerin is as follows. Once the optimal dose of aerosolized rottlerin in OVA and HDM murine asthma models and controls (PBS sensitized) is identified, this dose will be administered after the induction of asthma (vs. administration of rottlerin during the asthma induction protocol). In these experiments, rottlerin treatment will be initiated after 3 weeks of OVA sensitization and after 5 weeks of HDM sensitization. OVA and HDM challenges will be continued and animals will be treated for 1-3 weeks with the optimal dose of aerosolized rottlerin. AHR will be determined prior to sacrifice. Histological analyses of inflammation and airway remodeling, and BAL cell counts will be performed. Serum and tissue samples will be obtained to determine the rottlerin drug levels in these aerosolized treated animals using previously reported techniques (Varma et al., “Oral contraptive—Part III. Further observations on the antifertility effect of rottlerin” Indian J Physiol Pharmacol. Vol. 3: p. 168-72 (1959)). If for any reason aerosolized rottlerin is not efficacious, the dosing experiments will be performed using systemic administration.

The duration of action of a single administration of aerosolized rottlerin will be determined by measuring the R_{L} in response to MCh at 5, 30, 60, 120 minutes and 6 and 12 hours following the drug administration to determine duration of action of rottlerin in the OVA and HDM mouse asthma models.

### Example 8

### Rottlerin Derivatives

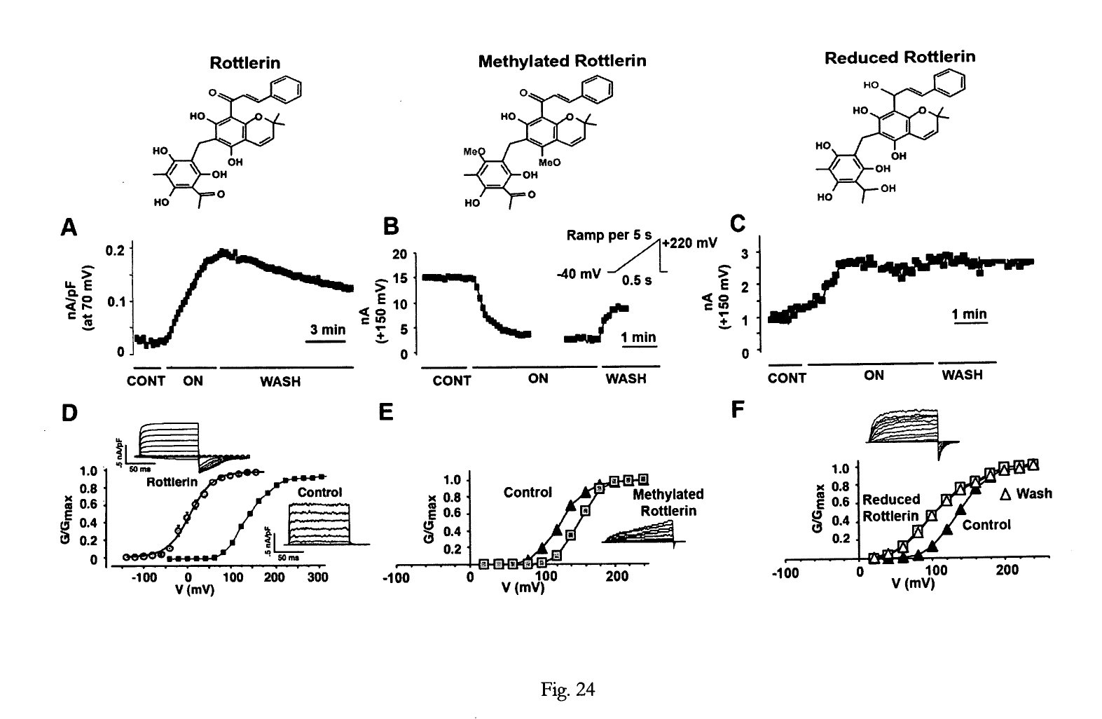

One approach to understand the mechanism by which rottlerin attenuates AHR and inflammation is to dissociate the two effects by developing rottlerin derivatives, some of which may lack BK modulatory properties or lack anti-inflammatory properties. Two derivatives, reduced rottlerin and methylated rottlerin, are shown in FIG. 24. Methylated rottlerin is an inhibitor of BK channel, whereas reduced rottlerin is an activator, albeit less potent than rottlerin, of BK channels. Methylated rottlerin significantly slows activation kinetics (time for opening) of the channel and shifted the G-V curve to the right compared to control (FIG. 24E). In contrast, reduced rottlerin shifts the G-V curve to the left compared to control (FIG. 24F), although the shift is significantly less than rottlerin (FIG. 24D).

Rottlerin derivatives will be compared with rottlerin. The ones with higher specificity for BK channels (i.e. BK activating properties without effects on other ion channels or on T cells) and ones with higher specificity for T-cell suppression (i.e. no effect on BK channels or other ion channels) will be identified. In addition to specificity, derivatives with higher potency for either or both of the two activities will also be identified. The most promising derivatives in terms of specificity and/or potency will be advanced to animal studies to evaluate the separate therapeutic contributions of BK activation and T-cell suppression in the application of rottlerin and its derivatives to the alleviation of asthma. In this way, the basis for the alleviation of asthma by rottlerin and its derivatives will be better understood and important therapeutic lead compounds will be found.

In testing rottlerin derivatives for inducing membrane hyperpolarization via activation of BK channels, the Molecular Devices FLIPR system and the membrane potential assay kit (cat #R8034) (Molecular Devices, Sunnyvale, Calif.) will be used. The system has been used to perform high throughput screening of K⁺ channel activators (Vasilyev et al., A novel high-throughput screening assay for HCN channel blocker using membrane potential-sensitive dye and FLIPR. J Biomol Screen, 14(9): p. 1119-28 (2009)). The assay is based on the use of fluorescent dyes, which accumulate inside cells upon depolarization of the membrane potential, leading to elevated fluorescence. Hyperpolarization of the membrane potential leads to reduced fluorescence. HEK cells stably expressing human BK channels will be plated at 2.6×10⁴ cells/well (96-well plate)—this plating density results in full confluency of cells in the plate 24 hours post-plating. Cell viability will be confirmed by propidium iodide exclusion. Growth media will be removed from the microplate using a Microplate Washer (BioTek Instruments Inc., Winooski, Vt.) and the cells washed with Hank's balanced salt saline (HBSS). The cells will be loaded with the membrane potential sensitive dye at room temperature for 30 minutes. The solution will be aspirated leaving only residual volume and the plates positioned within the FLIPR reading chamber. Background fluorescence will be monitored for 14 seconds followed by a single step addition of rottlerin or its derivatives, in varying concentrations. The fluorescence response will be captured for 5 minutes in 2-second intervals. At the conclusion of the 5-minute period, iberiotoxin, a specific BK channel inhibitor, will be added. For all experiments, incubation with iberiotoxin will also be included during the dye loading step as a control. In these controls, no change in fluorescence should be observed upon addition of rottlerin or its derivatives. Data will be analyzed using FLIPR software, in which % reduction in fluorescence (which is associated with membrane hyperpolarization) will be plotted against the respective drug concentrations (Vasilyev et al., A novel high-throughput screening assay for HCN channel blocker using membrane potential-sensitive dye and FLIPR. J Biomol Screen, 14(9): p. 1119-28 (2009)) and compared to rottlerin.

Whether rottlerin derivatives activate hERG, leading to membrane hyperpolarization will be determined. A similar approach using voltage-sensitive dyes has been previously used to determine hERG channel inhibition (Dorn et al., Evaluation of a high-throughput fluorescence assay method for HERG potassium channel inhibition. J Biomol Screen, 10(4): p. 339-47 (2005)). A CHO-hERG stable cell line will be used. The voltage sensitive oxonol dyes, such as DiBAC₄, at concentrations of 10 nM and higher significantly increase activity of BK channels in the presence of the β1 subunits (Morimoto et al., Voltage-sensitive oxonol dyes are novel large-conductance Ca²⁺-activated K⁺ channel activators selective for β1 and β4 but not for β2 subunits. Mol Pharmacol, 71(4): p. 1075-88 (2007)). BK α subunit alone, however, is not affected by up to 1 μM DiBAC₄. Thus, since rottlerin's activation of BK channels is not dependent upon β subunits, the derivatives will be tested with the FLIPR system on channels composed of BK α subunit only.

Rottlerin derivatives will be tested for BK channel function (electrophysiology and tracheal rings). For those compounds with comparable EC₅₀ as rottlerin, cellular electrophysiology studies will be perform using patch clamp of BK α subunit-expressing-HEK cells (stable line) and acutely isolated tracheal smooth muscle. The effects of the rottlerin derivatives on the rates of opening and closing of the channel and conductance-voltage relationship will be studied (Zakharov et al., Activation of the BK (SLO1) potassium channel by mallotoxin. J Biol Chem, 280(35): p. 30882-7 (2005)). For electrophysiological testing in isolated ASM, physiological changes in BK channel activity are assessed by measuring spontaneous transient outward currents (STOCs) in non-dialysed cells by perforated patch-clamp recordings. STOCs are BK channel openings caused by instantaneous ryanodine receptor openings. STOC amplitude represents the number of BK channels opening after a spark event (Zhuge et al., Spontaneous transient outward currents arise from microdomains where BK channels are exposed to a mean Ca²⁺ concentration on the order of 10 microM during a Ca²⁺ spark. J Gen Physiol, 2002. 120(1): p. 15-27). STOCs (perforated patch, voltage steps from −70 mV, stepped at 10 second intervals to +30 mV) will be measured in isolated tracheal ASMC.

Rottlerin derivatives will also be tested for relaxation of murine tracheal rings. A similar approach as the one in Example 4 (see also FIG. 13), in which rottlerin enhanced isoproterenol-mediated relaxation in trachea derived from both control and OVA-sensitized/challenged animals, will be taken.

An important finding is the marked diminution of inflammatory cells in the BAL fluid and in the peribronchial and perivascular space in the rottlerin-treated asthmatic mice as compared to PBS-treated asthmatic mice. The inventors have found that rottlerin significantly decreases the Th2 cytokines, IL-4 IL-5 and IL-13 in the BAL fluid of asthmatic mice. Rottlerin is known to inhibit human T cell responses (Springael et al., Rottlerin inhibits human T cell responses. Biochem Pharmacol, 73(4): p. 515-25 (2007)), and PMA-induced phosphorylation of Erk-1 and Erk-2 in Jurkat T cells and purified human CD4+ T cells from peripheral blood (Roose et al., A diacylglycerol-protein kinase C-RasGRP1 pathway directs Ras activation upon antigen receptor stimulation of T cells. Mol Cell Biol, 25(11): p. 4426-41 (2005)).

Thus, rottlerin and its derivatives will be tested for immunological effect using an in vitro lymphocyte assay. The OVA-specific responses in thoracic lymph node cultures will be examined as previously described (Bao et al., A novel antiinflammatory role for andrographolide in asthma via inhibition of the nuclear factor-kB pathway. Am J Respir Crit Care Med, 179: p. 657-665 (2009)). The thoracic lymph nodes will be harvested from mice 24 hours (Lai et al, The role of sphingosine kinase in a murine model of allergic asthma. J Immunol, 180: p. 4323-4329 (2008)) after the last OVA aerosol challenge. Lymph node cultures will be exposed to 200 μg/ml OVA for 72 hours in the absence or presence of rottlerin (doses 0.1, 1, 10, 50 μM) or derivatives. The levels of IL-4, IL-5 and IFN-γ in culture supernatant will be determined using ELISA.

Certain derivatives will be further studied in vivo to assess the relative contributions of BK channel activation and anti-inflammatory effects to the rottlerin-induced reduction in AHR observed in the OVA-asthma model. The derivatives to be studied in vivo will be categorized based upon their efficacy as a BK channel agonist (without anti-inflammatory effects), BK channel agonist with anti-inflammatory effects and an anti-inflammatory effects without BK channel activating properties. Selected derivatives administered, systemically or via nebulization, acutely or over an extended period, will be studied.

The derivatives will be compared to rottlerin and PBS in the mice subjected to the 24 day OVA-asthma model as described above, to determine whether specific BK channel agonists can acutely dilate hypercontractile airway and to dissect the molecular mechanisms underlying the rottlerin-induced attenuation in airway hyperreactivity in the murine asthma model. The derivatives will be injected I.P. every other day and airway resistance will be determined, in response to methacholine at day 24. Cell counts and analysis of cytokines (levels of Th-2 type cytokines and Th-1 type cytokine, IFN-γ,) will be determined in peripheral blood and BAL fluid.

The following methodologies may be used to perform the experiments outlined above.

### Smooth Muscle Cell Isolation

Mice will be euthanized by injection of sodium pentobarbital, trachea removed and transferred to ice-cold-low Ca²⁺ physiological saline solution (PSS). After the removal of epithelium, cartilage and connective tissue, the trachealis muscle will be minced and placed in PSS containing papain, DTT and bovine serum albumin at 37° C. for 20 minutes, followed by PSS containing collagenase H, collagenase II, DTT and BSA at 37° C. for 30 minutes. The digested tissue will be washed, and single cells released by gentle trituration with a fire-polished glass pipette.

### Cellular Electrophysiology

Spontaneous BK currents will be measured using the whole-cell patch clamp technique in the amphotericin B (250 μg/ml) perforated patch configuration as described by Santana and colleagues (Amberg et al., Modulation of the molecular composition of large conductance, Ca²⁺ activated K⁺ channels in vascular smooth muscle during hypertension. J Clin Invest, 112(5): p. 717-24 (2003); Amberg, G. C. and L. F. Santana, Downregulation of the BK channel beta1 subunit in genetic hypertension. Circ Res, 93(10): p. 965-71 (2003)). Cells will be continuously superfused with normal Tyrode's solution. ASMC will be held at −40 mV. Petri dishes with ASMC cells will be mounted on the stage of an inverted microscope, which will serve as a perfusion chamber. Experimental solutions will be applied by local perfusion.

### In Vivo Measurement of Airway Hyperreactivity (AHR)

Two days after the last aerosol, AHR will be measured by invasive restrained whole body plethysmography (rWBP) (BUXCO Electronics) in response to inhaled methacholine (Sigma, St. Louis, Mo.). For dynamic lung resistance measurements will be performed using the PLY3011 chamber (BUXCO). Mice will be anesthetized with a cocktail of 25 mg per kg body weight ketamine hydrochloride (Bioniche Pharma USA LLC, Lake Forest, Ill.) and 2.5 mg per kg body weight xylazine (Lloyd Labs, Quezon City, Philippines), tracheotomized, and immediately intubated with an 18-gauge catheter, followed by mechanical ventilation (Columbus Instruments, Columbia, Ohio). Respiratory frequency will be set at 150 breaths/minute with a tidal volume of 0.2 ml. Increasing concentrations of methacholine (0-50 mg/ml) will be administered at the rate of 20 puffs per 10 seconds, with each puff of aerosol delivery lasting 10 ms, via a nebulizer aerosol system with a 4-6 μm aerosol particle size generated by a nebulizer head (Aeroneb, Aerogen Ltd., Galway, Ireland). The nebulizer will be attached to a small aerosol block, which is placed in the inspiratory flow line. In this way the aerosol will be injected directly into the airway. Baseline resistance will be restored before administration of the subsequent doses of methacholine. The flow and pressure signals will be measured and processed together to determine resistance and compliance using BioSystem XA software (BUXCO).

### Other OVA Models

The intermediate and long-term OVA models may also be used to explore the efficacy of rottlerin and rottlerin derivative.

All documents cited above are incorporated by reference as if recited in full herein.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

What is claimed is:

1. A method of treating or ameliorating the effects of a disease characterized by altered smooth muscle contractility comprising administering to a patient suffering from such a disease an effective amount of a large-conductance Ca²⁺-activated K⁺ (BK) channel modulator.

2. The method according to claim 1, wherein the BK channel modulator is a BK channel activator.

3. The method according to claim 2, wherein the BK channel activator is selected from the group consisting of rottlerin, flindokalner (Bristol-Myers Squibb), BMS-554216 (Bristol-Myers Squibb), Pharmaprojects No. 4420 (Merck & Co), Pharmaprojects No. 4494 (Merck & Co), NS-1619 (NeuroSearch), NSD-551 (NeuroSearch), NS-8 (Nippon Shinyaku), a pharmaceutically acceptable salt thereof, and combinations thereof.

4. The method according to claim 3, wherein the BK channel activator is rottlerin.

5. The method according to claim 4, wherein the rottlerin is in the form of an extract from Mallotus phillippinensis.

6. The method according to claim 1, wherein the disease is selected from the group consisting of asthma, chronic obstructive pulmonary disease, urinary incontinence, and hypertension.

7. The method according to claim 1, wherein the disease is asthma.

8. The method according to claim 1, wherein the disease is chronic.

9. The method according to claim 1, wherein the disease is acute.

10. The method according to claim 1, wherein the BK channel modulator is administered as part of a pharmaceutical composition.

11. The method according to claim 10, wherein the pharmaceutical composition is in a unit dosage form.

12. The method according to claim 11, wherein the unit dosage form is inhaled.

13. The method according to claim 10, wherein the pharmaceutical composition is co-administered with a composition selected from the group consisting of corticosteroids, anti-cholinergics, anti-leukotrienes, β-agonists, and phosphodiesterase inhibitors.

14. The method according to claim 13, wherein the pharmaceutical composition is co-administered with a β-agonist.

15. A method of treating or ameliorating the effects of asthma comprising administering to a patient suffering from asthma an effective amount of a BK channel modulator.

16. The method according to claim 15, wherein the BK channel modulator is administered as part of a pharmaceutical composition.

17. The method according to claim 16, wherein the pharmaceutical composition is in a unit dosage form.

18. The method according to claim 17, wherein the unit dosage form is inhaled.

19. The method according to claim 16, wherein the pharmaceutical composition is co-administered with a composition selected from the group consisting of corticosteroids, anti-cholinergics, anti-leukotrienes, β-agonists, and phosphodiesterase inhibitors.

20. The method according to claim 19, wherein the pharmaceutical composition is co-administered with a β-agonist.

21. A method for decreasing airway constriction and/or airway resistance in a patient without increasing the heart rate of the patient comprising administering to the patient an effective of amount of a BK channel modulator or a pharmaceutical composition comprising a pharmaceutically acceptable carrier and a BK channel modulator.

22. The method according to claim 21, wherein the pharmaceutical composition is in a unit dosage form.

23. The method according to claim 22, wherein the unit dosage form is inhaled.

24. The method according to claim 21, wherein the pharmaceutical composition is co-administered with a composition selected from the group consisting of corticosteroids, anti-cholinergics, anti-leukotrienes, β-agonists, and phosphodiesterase inhibitors.

25. The method according to claim 24, wherein the pharmaceutical composition is co-administered with a β-agonist.

26. A method for modulating inflammation in a lung of a patient, the method comprising administering to a patient an effective of amount of a BK channel modulator or a pharmaceutical composition comprising a pharmaceutically acceptable carrier and a BK channel modulator, which amount is sufficient to modulate the inflammation.

27. The method according to claim 26, wherein the modulation is a decrease in inflammation.

28. The method according to claim 26, wherein the pharmaceutical composition is in a unit dosage form.

29. The method according to claim 28, wherein the unit dosage form is inhaled.

30. The method according to claim 26, wherein the pharmaceutical composition is co-administered with a composition selected from the group consisting of corticosteroids, anti-cholinergics, anti-leukotrienes, β-agonists, and phosphodiesterase inhibitors.

31. The method according to claim 30, wherein the pharmaceutical composition is co-administered with a β-agonist.

32. A pharmaceutical composition for treating or ameliorating the effects of a disease characterized by altered smooth muscle contractility, the composition comprising a pharmaceutically acceptable carrier and a BK channel modulator.

33. The pharmaceutical composition according to claim 32, wherein the disease is selected from the group consisting of asthma, chronic obstructive pulmonary disease, urinary incontinence, and hypertension.

34. The pharmaceutical composition according to claim 32, wherein the disease is asthma.

35. The pharmaceutical composition according to claim 32, which is in a unit dosage form.

36. The pharmaceutical composition according to claim 35, wherein the unit dosage form is inhaled.

37. The pharmaceutical composition according to claim 32, which is co-administered with a composition selected from the group consisting of corticosteroids, anti-cholinergics, anti-leukotrienes, β-agonists, and phosphodiesterase inhibitors.

38. The method according to claim 37, wherein the pharmaceutical composition is co-administered with a β-agonist.

39. A pharmaceutical composition for treating or ameliorating the effects of asthma, the composition comprising a pharmaceutically acceptable carrier and a BK channel modulator.
